(19) United States
(12) Patent Application Publication
(10) Pub. No.: US 2022/0008526 A1
(43) Pub. Date: Jan. 13, 2022

(54) METHODS AND COMPOSITIONS FOR CANCER IMMUNOTHERAPY

(71) Applicant: Torque Therapeutics, Inc., Cambridge, MA (US)

(72) Inventors: Thomas Lars Andresen, Vanlose (DK); Douglas Jones, Cambridge, MA (US); Elena Geretti, Cambridge, MA (US); Gulzar Ahmad, Cambridge, MA (US)

(21) Appl. No.: 17/293,995

(22) PCT Filed: Nov. 15, 2019
(86) PCT No.: PCT/US2019/061837

§ 371 Date: May 14, 2021

Related U.S. Application Data
(60) Provisional application No. 62/930,399, filed on Nov. 4, 2019.
(60) Provisional application No. 62/825,496, filed on Mar. 28, 2019.
(60) Provisional application No. 62/767,515, filed on Nov. 15, 2018.

(51) Int. Cl. A61K 39/00; C07K 14/54; C07K 16/28

(52) U.S. Cl. CPC A61K 39/00114; A61K 39/001139; A61K 2039/5156; C07K 16/28; C07K 14/54

(57) ABSTRACT

Disclosed herein are compositions and methods for cancer immunotherapy, and more particularly immune cells loaded with protein clusters and/or immunostimulatory fusion molecules (IFMs), in combination with an inhibitor of a checkpoint inhibitor.

### CROSS REFERENCE TO RELATED APPLICATIONS

This application claims priority to and the benefit of U.S. Provisional Application Nos. 62/767,515 filed Nov. 15, 2018, 62/825,496 filed Mar. 28, 2019, and 62/930,399 filed Nov. 4, 2019, the disclosures of each of which applications are hereby incorporated herein by reference in their entirety.

### FIELD

Methods and compositions disclosed herein relate to cancer immunotherapy, in particular preparation and use of antigen-specific T lymphocytes for immune cell therapies.

### BACKGROUND

Immune cell therapies, e.g., adoptive cell therapy (ACT), include the steps of collecting immune cells from a subject, expanding the cells, and reintroducing the cells into the same subject or a different subject. For example, ACT of donor-derived, ex-vivo expanded human antigen-specific, cytotoxic T lymphocytes (CTLs) has emerged as a promising approach to treat cancer. Other ACT includes cultured tumor infiltrating lymphocytes (TILs), isolated and expanded T cell clones, and genetically engineered lymphocytes (e.g., T cells) that express conventional T cell receptors or chimeric antigen receptors. The genetically engineered lymphocytes are designed to eliminate cancer cells expressing specific antigen(s) and are expanded and delivered to a patient. ACT can provide tumor specific lymphocytes (e.g., T cells) that lead to a reduction in tumor cells in a patient.

However, the anti-tumor activity of T cell therapies has been limited by insufficient T cell expansion and by checkpoint immunosuppression. Thus, a need exists for improved compositions and methods that can overcome these limitations.

### SUMMARY

Disclosed herein are improved methods and compositions for T cell therapies. More particularly, disclosed herein are T cells surface modified to carry engineered backpacks, in combination with inhibitors of checkpoint inhibitor (e.g., an anti-PD-L1 antibody). In some embodiments, the backpacks can contain a plurality of therapeutic protein monomers (e.g., cytokines such as IL-15) reversibly cross-linked by biodegradable linkers. In other embodiments, the backpacks can be fusion proteins designed to tether to immune cell surface.

In one aspect, disclosed herein is a therapeutic (e.g., cancer immunotherapy) composition comprising:

a nucleated cell loaded with a plurality of protein clusters and/or immunostimulatory fusion molecules (IFMs); and

an inhibitor of a checkpoint inhibitor.

In some embodiments, the protein clusters can each include a plurality of therapeutic protein monomers reversibly cross-linked to one another via a plurality of biodegradable cross-linkers, wherein the protein cluster has a size between 30 nm and 1000 nm in diameter measured by dynamic light scattering, wherein the cross-linker degrades, after administration into a subject in need thereof, under physiological conditions so as to release the therapeutic protein monomers from the protein cluster, wherein optionally the protein cluster further comprises a surface modification such as polycation so as to allow the protein cluster to associate with the nucleated cell.

In some embodiments, the therapeutic protein monomers can include one or more cytokine molecules and optionally one or more costimulatory molecules, wherein:
(i) the one or more cytokine molecules are selected from IL-15, IL-2, IL-7, IL-10, IL-12, IL-18, IL-21, IL-23, IL-4, IL-1alpha, IL-1beta, IL-5, IFNgamma, TNFa, IFNalpha, IFNbeta, GM-CSF, or GCSF; and
(ii) the one or more costimulatory molecules are selected from CD137, OX40, CD28, GITR, VISTA, anti-CD40 antibody, or CD3.

In some embodiments, the cross-linker can be a degradable or hydrolysable linker. In some embodiments, the degradable linker is a redox responsive linker. Exemplary linkers as well as methods of making and using various linkers (e.g., to make nanogels or backpacks) are disclosed in PCI Application No. PCT/US2018/049594, U.S. Publication No. 2017/0080104, U.S. Pat. No. 9,603,944, and U.S. Publication No. 2014/0081012, each of which is incorporated herein by reference in its entirety.

In certain embodiments, each IFM can be engineered to contain an immunostimulatory cytokine molecule and a targeting moiety (e.g., an antibody or an antigen-binding fragment thereof) having an affinity to an antigen on the surface of the nucleated cell, wherein the immunostimulatory cytokine molecule is operably linked to targeting moiety. Exemplary IFMs (also referred to as “tethered fusion” or TF) are disclosed in PCT International Publication Nos. WO 2019/010219 and WO 2019/010222, each incorporated herein by reference in its entirety.

In some embodiments, the immunostimulatory cytokine molecule is selected from one or more of IL-15, IL-2, IL-6, IL-7, IL-12, IL-18, IL-21, IL-23, or IL-27 or variant forms thereof. The antigen can be selected from one or more of CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR.

In one embodiment, the IFM contains IL-12, e.g., single-chain IL-12p70 fused to a humanized anti-CD45 Fab. The single-chain IL-12p70 can contain IL-12B and IL-12A joined by flexible linker.

In various embodiments, the nucleated cell and the inhibitor of checkpoint inhibitor can be provided and administered separately (e.g., sequentially) to a patient in need of, e.g., cancer immunotherapy. In some embodiments the nucleated cell can be from a population of T cells that have been enriched or trained to possess specificity against one or more tumor-associated antigens (TAAs).

In certain embodiments, the checkpoint inhibitor can be one or more of PD-1, PD-L1, LAG-3, TIM-3, or CTLA-4. The inhibitor of the checkpoint inhibitor can be an antibody or antigen-binding fragment thereof that binds and neutralizes or inhibits the checkpoint inhibitor.

Also provided herein is a method for providing cancer immunotherapy, comprising:

administering to a patient in need thereof a plurality of nucleated cells loaded with a plurality of protein clusters and/or IFMs; and

administering to the patient an inhibitor of a checkpoint inhibitor.

### BRIEF DESCRIPTION OF THE DRAWINGS

FIG. 1 illustrates an exemplary DEEP™ IL-15 preparation.

FIG. 2: IL-15 increases PD-1 expression on PMEL T cells.

FIG. 3: Experimental outline for DEEP™ IL-15 primed PMEL and anti-PD-L1 combination Study 1.

FIG. 4: Study 1 shows that αPD-L1 improves the anti-tumor activity of DEEP™-15 Primed PMEL T cells in the presence of lymphodepletion (CPX).

FIG. 5: Study 1 shows that DEEP™ IL-15 Primed PMEL T cells in combination with αPD-L1 increase anti-tumor activity, increase tumor free survivors (TFS), result in no changes in exposure to IL15-Fc, and are well tolerated, with no changes in body weight.

FIG. 6: Experimental outline for DEEP™ IL-15 primed PMEL and anti-PD-L1 combination Study 2.

FIG. 7: DP-15 PMEL+a-PD-L1 shows improved anti-tumor activity and survival compared to PMEL+αPD-L1.

FIG. 8: Study 2 shows that DEEP′ IL-15 Primed PMEL T cells in combination with αPD-L1 increase anti-tumor activity, increase tumor free survivors (TFS), result in no changes in exposure to IL15-Fc, and are well tolerated, with no changes in body weight.

FIG. 9: Experimental outline for DEEP™ IL-15 primed PMEL and anti-PD-L1 combination Study 3.

FIG. 10: Combination with αPD-L1 results in increased PMEL T cells activation (IFN-γ secretion) in the tumor.

FIG. 11: Survival analysis of tumor-bearing mice treated with tumor-specific T cells tethered with IL-12 TF with or without combination with PD-L1 blockade. Arrows indicate days of adoptive transfer of the tumor-specific cell therapy.

FIG. 12: IFNg and PD-L1 concentration in the tumor.

### DETAILED DESCRIPTION

Cancer immunotherapy, including adoptive T cell therapy, is a promising strategy to treat cancer because it harnesses a subject's own immune system to attack cancer cells. Nonetheless, a major limitation of this approach is the rapid decline in viability and function of the transplanted T lymphocytes. In order to maintain high numbers of viable tumor-specific cytotoxic T lymphocytes in tumors, co-administration of immunostimulatory agents with transferred cells is necessary. When given systemically at high doses, these agents could enhance the in vivo viability of transferred (i.e., donor) cells, improve the therapeutic function of transferred cells, and thus lead to overall improved efficacy against cancer; however, high doses of such agents could also result in life-threatening side effects. For example, the use of interleukin-2 (IL-2) as an adjuvant greatly supports adoptive T cell therapy of melanoma, where IL-2 provides key adjuvant signals to transferred T cells but also elicits severe dose-limiting inflammatory toxicity and expands regulatory T cells (Tregs). One approach to focus adjuvant activity on the transferred cells is to genetically engineer the transferred cells to secrete their own supporting factors. The technical difficulty and challenges as well as the high cost for large-scale production of genetically engineered T lymphocytes have significantly limited the potential of this method in clinical applications, to date.

Disclosed herein, in some aspects, is a combination therapy of inhibitor of checkpoint inhibitor and immune cells loaded with therapeutic protein clusters or tethered fusions. This permits simple, safe and efficient delivery of biologically-active agents, such as a drug, protein (e.g., adjuvants such as IL-2) or particle to cells onto the plasma membrane of target immune cells, while overcoming checkpoint immunosuppression commonly associated with such cell therapy. In certain embodiments, such therapeutic protein cluster or tethered fusion composition is referred to as “nanogel,” “nanoparticle,” or “backpack,” which terms are used interchangeably herein. For clarity, a “backpack” may refer to a cluster of monomers (e.g., IL15-Fc heterodimers) cross-linked together, or a tethered fusion (TF) molecule (e.g., IL-12-TF such as IL-12 and anti-CD45 antibody fusion). The composition can be loaded, anchored, tethered or backpacked (used interchangeably) onto cells, e.g., nucleated cells. The loading process is also referred to as “priming” and the resulting cells can be referred to as “primed” cells. Backpacked or loaded or primed cells can have many therapeutic applications. For example, loaded T cells can be used in T cell therapies including ACT (adoptive cell therapy). Other important immune cell types can also be loaded, including for example, B cells, tumor infiltrating lymphocytes, NK cells, antigen-specific CD8+ T cells, T cells genetically engineered to express chimeric antigen receptors (CARs) or CAR-T cells, T cells genetically engineered to express T-cell receptors specific to an tumor antigen, tumor infiltrating lymphocytes (TILs), and/or antigen-trained T cells (e.g., T cells that have been “trained” by antigen presenting cells (APCs) displaying antigens of interest, e.g. tumor associated antigens (TAA)).

Unexpectedly, it has been surprisingly discovered that in the presence of lymphodepletion, anti-PD-L1 further improves the anti-tumor activity of DEEP™ IL-15 (a multimer of chemically crosslinked IL-15/IL-15 Ra/Fc heterodimers (IL15-Fc)) or DEEP™ IL-12 (single-chain IL-12p70 fused to a humanized anti-CD45 Fab) primed cells in a synergistic manner. That is, there is a statistically significant difference (increase) in the efficacy of the combination of anti-PD-L1 and DEEP™ IL-15 or DEEP™ IL-12, compared to what would have been expected if the efficacy were purely additive.

In addition to the foregoing, the present disclosure further contemplates other nanostructures that comprise other protein therapeutics for purposes other than adjuvant effect on adoptively-transferred cells. Those of skill in the art will readily recognize that the disclosure has broader applications, as provided herein.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

The term “therapeutic,” “therapeutic agent,” “active,” “active agent,” “active pharmaceutical agent,” “active drug” or “drug” as used herein means any active pharmaceutical ingredient (“API”), including its pharmaceutically acceptable salts (e.g. the hydrochloride salts, the hydrobromide salts, the hydroiodide salts, and the saccharinate salts), as well as in the anhydrous, hydrated, and solvated forms, in the form of prodrugs, and in the individually optically active enantiomers of the API as well as polymorphs of the API. Therapeutic agents include pharmaceutical, chemical or biological agents. Additionally, pharmaceutical, chemical or biological agents can include any agent that has a desired property or affect whether it is a therapeutic agent. For example, agents also include diagnostic agents, biocides and the like.

The terms “protein”, “peptide” and “polypeptide” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures. It should be understood that the term “protein” includes fusion or chimeric proteins, as well as cytokines, antibodies and antigen-binding fragments thereof.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule. “Bispecific antibody molecule” as used herein refers to an antibody molecule that has specificity for more than one (e.g., two, three, four, or more) epitope and/or antigen.

“Antigen” (Ag) as used herein refers to a macromolecule, including all proteins or peptides. In some embodiments, an antigen is a molecule that can provoke an immune response, e.g., involving activation of certain immune cells and/or antibody generation. Antigens are not only involved in antibody generation. T cell receptors also recognized antigens (albeit antigens whose peptides or peptide fragments are complexed with an MHC molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chotia control so that the frameworks have consistent numbering across antibodies.

In some embodiments, the antigen-binding fragment of an antibody (e.g., when included as part of a fusion molecule) can lack or be free of a full Fc domain. In certain embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

As used herein, a “cytokine” or “cytokine molecule” refers to full length, a fragment or a variant of a naturally-occurring, wild type cytokine (including fragments and functional variants thereof having at least 10% of the activity of the naturally-occurring cytokine molecule). In embodiments, the cytokine molecule has at least 30, 50, or 80% of the activity, e.g., the immunomodulatory activity, of the naturally-occurring molecule. In certain embodiments, cytokines include interleukins (e.g., IL-2, IL-7, IL-15, IL-10, IL-18, IL-21, IL-23, IL-12, IFN-gamma, IFN-alpha, GM-CSF, FLT3-ligand, or superagonist/mutant forms of these cytokines such as IL-15SA). In embodiments, the cytokine molecule further comprises a receptor domain, e.g., a cytokine receptor domain, optionally, coupled to an immunoglobulin Fc region. In other embodiments, the cytokine molecule is coupled to an immunoglobulin Fc region. In other embodiments, the cytokine molecule is coupled to an antibody molecule (e.g., an immunoglobulin Fab or scFv fragment, a Fab fragment, a FAB₂ fragment, or an affibody fragment or derivative, e.g., a sdAb (nanobody) fragment, a heavy chain antibody fragment, single-domain antibody, a bi-specific or multispecific antibody), or non-antibody scaffolds and antibody mimetics (e.g., lipocalins (e.g., anticalins), affibodies, fibronectin (e.g., monobodies or Adnectins), knottins, ankyrin repeats (e.g., DARPins), and A domains (e.g., avimers)).

As used herein, an “immunostimulatory fusion molecule” (IFM; used interchangeably with “tethered fusion”) refers to a chimeric molecule comprising an immune stimulating moiety and an immune cell targeting moiety. The immune cell targeting moiety can include an antibody or an antigen-binding fragment thereof, having an affinity to an antigen on the surface of a target immune cell. The immunostimulatory cytokine molecule can be operably linked to the antibody or antigen-binding fragment thereof, e.g., via a linker. Exemplary tethered fusion protein (e.g., IL-15 tethered fusion and IL-12 tethered fusion) such as those disclosed in PCT International Application Nos. PCT/US2018/040777, PCT/US2018/040783 and PCT/US2018/040786, all incorporated herein by reference.

As used herein, “administering” and similar terms mean delivering the composition to an individual being treated. Preferably, the compositions of the present disclosure are administered by, e.g., parenteral, including subcutaneous, intramuscular, or preferably intravenous routes.

As used herein, the terms “cancer” and “cancerous” refer to or describe the physiological condition in mammals that is typically characterized by unregulated cell growth. Examples of cancer include, but are not limited to, melanoma, carcinoma, lymphoma, blastoma, sarcoma, and leukemia or lymphoid malignancies. More particular examples of cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

“Nucleated cells” are cells which contain nucleus. In some embodiments, the nucleated cells can be immune cells such as immune effector cells.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. The term “immune cell” includes immune effector cells described herein. “Immune cell” also refers to modified versions of cells involved in an immune response, e.g. modified NK cells, including NK cell line NK-92 (ATCC cat. No. CRL-2407), haNK (an NK-92 variant that expresses the high-affinity Fc receptor FcγRIIIa (158V)) and taNK (targeted NK-92 cells transfected with a gene that expresses a CAR for a given tumor antigen), e.g., as described in Klingemann et al. supra.

“Immune effector cell,” as that term is used herein, refers to a cell that is involved in an immune response, e.g., in the promotion of an immune effector response. Examples of immune effector cells include, but are not limited to, T cells, e.g., CD4+ T cells, CD8+ T cells, alpha T cells, beta T cells, gamma T cells, and delta T cells; B cells; natural killer (NK) cells; natural killer T (NKT) cells; dendritic cells; and mast cells. In some embodiments, the immune cell is an immune cell (e.g., T cell or NK cell) that comprises, e.g., expresses, a Chimeric Antigen Receptor (CAR), e.g., a CAR that binds to a cancer antigen. In other embodiments, the immune cell expresses an exogenous high affinity Fc receptor. In some embodiments, the immune cell comprises, e.g., expresses, an engineered T-cell receptor. In some embodiments, the immune cell is a tumor infiltrating lymphocyte. In some embodiments the immune cells comprise a population of immune cells and comprise T cells that have been enriched for specificity for a tumor-associated antigen (TAA), e.g., enriched by sorting for T cells with specificity towards MHCs displaying a TAA of interest, e.g. MART-1. In some embodiments immune cells comprise a population of immune cells and comprise T cells that have been “trained” to possess specificity against a TAA by an antigen presenting cell (APC), e.g., a dendritic cell, displaying TAA peptides of interest. In some embodiments, the T cells are trained against a TAA chosen from one or more of MART-1, MAGE-A4, NY-ESO-1, SSX2, Survivin, or others. In some embodiments the immune cells comprise a population of T cells that have been “trained” to possess specificity against multiple TAAs by an APC, e.g. a dendritic cell, displaying multiple TAA peptides of interest. Such T cells are also referred to as multi-targeted T cells (“MTC”) herein. In some embodiments, the immune cell is a cytotoxic T cell (e.g., a CD8+ T cell). In some embodiments, the immune cell is a helper T cell, e.g., a CD4+ T cell.

“Tumor-associated antigen” (TAA) is an antigenic substance produced in tumor cells that triggers an immune response in the host. Tumor antigens are useful tumor markers in identifying tumor cells with diagnostic tests and are potential candidates for use in cancer therapy. In some embodiments, the TAA can be derived from, a cancer including but not limited to primary or metastatic melanoma, thymoma, lymphoma, sarcoma, lung cancer, liver cancer, non-Hodgkin's lymphoma, non-Hodgkins lymphoma, leukemias, uterine cancer, cervical cancer, bladder cancer, kidney cancer and adenocarcinomas such as breast cancer, prostate cancer, ovarian cancer, pancreatic cancer, and the like. TAAs can be patient specific. In some embodiments, TAAs may be p53, Ras, beta-Catenin, CDK4, alpha-Actinin-4, Tyrosinase, TRP1/gp75, TRP2, gpIOO, Melan-A/MART 1, Gangliosides, PSMA, HER2, WT1, EphA3, EGFR, CD20, MAGE, BAGE, GAGE, NY-ESO-1, Telomerase, Survivin, or any combination thereof. Exemplary TAAs include preferentially expressed antigen of melanoma (PRAME), synovial sarcoma X (SSX) breakpoint 2 (SSX2), NY-ESO-1, Survivin, and Wilms' tumor gene 1 (WT-1).

“Cytotoxic T lymphocytes” (CTLs) as used herein refer to T cells that have the ability to kill a target cell. CTL activation can occur when two steps occur: 1) an interaction between an antigen-bound MHC molecule on the target cell and a T cell receptor on the CTL is made; and 2) a costimulatory signal is made by engagement of costimulatory molecules on the T cell and the target cell. CTLs then recognize specific antigens on target cells and induce the destruction of these target cells, e.g., by cell lysis. In some embodiments, the CTL expresses a CAR. In some embodiments, the CTL expresses an engineered T-cell receptor.

As used herein, an “effective amount” means the amount of bioactive agent or diagnostic agent that is sufficient to provide the desired local or systemic effect at a reasonable risk/benefit ratio as would attend any medical treatment or diagnostic test. This will vary depending on the patient, the disease, the treatment being effected, and the nature of the agent.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions etc.

The term “treatment” or “treating” means administration of a drug for purposes including: (i) preventing the disease or condition, that is, causing the clinical symptoms of the disease or condition not to develop; (ii) inhibiting the disease or condition, that is, arresting the development of clinical symptoms; and/or (iii) relieving the disease or condition, that is, causing the regression of clinical symptoms.

The term “subject” includes living organisms in which an immune response can be elicited (e.g., mammals, human). In one embodiment, the subject is a patient, e.g., a patient in need of immune cell therapy. In another embodiment, the subject is a donor, e.g. an allogenic donor of immune cells, e.g., intended for allogenic transplantation.

The term “autologous” refers to any material derived from the same individual to whom it is later to be re-introduced into the individual.

The term, a “substantially purified cell” refers to a cell that is essentially free of other cell types and/or has been enriched relative to other cell types in the starting population. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other aspects, the cells are not cultured in vitro.

Various aspects of the disclosure are described in further detail below. Additional definitions are set out throughout the specification.

Monomers

Examples of protein monomers for use in accordance with the present disclosure include, without limitation, antibodies (e.g., IgG, Fab, mixed Fc and Fab), single chain antibodies, antibody fragments, engineered proteins such as Fc fusions, enzymes, co-factors, receptors, ligands, transcription factors and other regulatory factors, cytokines, chemokines, human serum albumin, and the like. These proteins may or may not be naturally occurring. Other proteins are contemplated and may be used in accordance with the disclosure. Any of the proteins can be reversibly modified through cross-linking to form a cluster or nanogel structure as disclosed in, e.g., U.S. Publication No. 2017/0080104, U.S. Pat. No. 9,603,944, U.S. Publication No. 2014/0081012, and PCT Application No. PCT/US17/37249 filed Jun. 13, 2017, all incorporated herein by reference.

In various embodiments, therapeutic protein monomers can be cross-linked using one or more cross-linkers disclosed herein. The therapeutic protein monomers can comprise one or more cytokine molecules and optionally one or more costimulatory molecules. Cytokine molecules can be selected from IL-15, IL-2, IL-7, IL-10, IL-12, IL-18, IL-21, IL-23, IL-4, IL-1alpha, IL-1beta, IL-5, IFNgamma, TNFa, IFNalpha, IFNbeta, GM-CSF, or GCSF. Costimulatory molecules are selected from CD137, OX40, CD28, GITR, VISTA, anti-CD40 antibody, or CD3.

In some embodiments, protein monomers of the disclosure are immunostimulatory proteins. As used herein, an immunostimulatory protein is a protein that stimulates an immune response (including enhancing a pre-existing immune response) in a subject to whom it is administered, whether alone or in combination with another protein or agent. Examples of immunostimulatory proteins that may be used in accordance with the disclosure include, without limitation, antigens, adjuvants (e.g., flagellin, muramyl dipeptide), cytokines including interleukins (e.g., IL-2, IL-7, IL-15, IL-10, IL-18, IL-21, IL-23 (or superagonist/mutant forms of these cytokines, such as, IL-15SA), IL-12, IFN-gamma, IFN-alpha, GM-CSF, FLT3-ligand), and immunostimulatory antibodies (e.g., anti-CTLA-4, anti-CD28, anti-CD3, or single chain/antibody fragments of these molecules). Other immunostimulatory proteins are contemplated and may be used in accordance with the disclosure. In some embodiments, the immunostimulatory proteins can be an antibody or antigen-binding fragment thereof that binds an inhibitor of an immunosuppressor, e.g., an inhibitor of a checkpoint inhibitor, such as PD-1, PD-L1, LAG-3, TIM-3, CTLA-4, inhibitory KIR, CD276, VTCN1, BTLA/HVEM, HAVCR2 and ADORA2A, e.g., as described in US 2016/0184399 incorporated herein by reference.

In some embodiments, protein monomers of the disclosure are antigens. Examples of antigens that may be used in accordance with the disclosure include, without limitation, cancer antigens, self-antigens, microbial antigens, allergens and environmental antigens. Other protein antigens are contemplated and may be used in accordance with the disclosure.

In some embodiments, proteins of the disclosure are cancer antigens. A cancer antigen is an antigen that is expressed preferentially by cancer cells (i.e., it is expressed at higher levels in cancer cells than on non-cancer cells) and, in some instances, it is expressed solely by cancer cells. Cancer antigens may be expressed within a cancer cell or on the surface of the cancer cell. Cancer antigens that may be used in accordance with the disclosure include, without limitation, MART-1/Melan-A, gp100, adenosine deaminase-binding protein (ADAbp), FAP, cyclophilin b, colorectal associated antigen (CRC)-0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4 and MAGE-05. The cancer antigen may be selected from the group consisting of GAGE-1, GAGE-2, GAGE-3, GAGE-4, GAGE-5, GAGE-6, GAGE-7, GAGE-8 and GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, PIA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20 and c-erbB-2. Other cancer antigens are contemplated and may be used in accordance with the disclosure.

In some embodiments, proteins of the disclosure are antibodies or antibody fragments including, without limitation, bevacizumab (AVASTIN®), trastuzumab (HERCEPTIN®), alemtuzumab (CAMPATH®, indicated for B cell chronic lymphocytic leukemia), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®) and Gliomab-H (indicated for brain cancer, melanoma). Other antibodies and antibody fragments are contemplated and may be used in accordance with the disclosure.

Proteins may be linked (e.g., covalently linked) to a degradable linker through any terminal or internal nucleophilic groups such as a —NH2 functional group (e.g., side chain of a lysine). For example, proteins can be contacted with a degradable linker under conditions that permit reversible covalent crosslinking of proteins to each other through the degradable linker. In some embodiments, the proteins can be cross-linked to form a plurality of protein nanogels. In some embodiments, the conditions include contacting the protein with the degradable linker in an aqueous buffer at a temperature of 4° C. to 25° C. In some embodiments, the contacting step can be performed in an aqueous buffer for 30 minutes to one hour. In some embodiments, the aqueous buffer comprises phosphate buffered saline (PBS). In some embodiments, the concentration of the protein in the aqueous buffer is 10 mg/mL, to 50 mg/mL (e.g., 10, 15, 20, 25, 30, 35, 40, 45 or 50 mg/mL).

### Cytokines

The methods and compositions, e.g., linker compounds, described herein can be used to cross-link one or more cytokine molecules. In embodiments, the cytokine molecule is full length, a fragment or a variant of a cytokine, e.g., a cytokine comprising one or more mutations. In some embodiments the cytokine molecule comprises a cytokine chosen from interleukin-1 alpha (IL-1 alpha), interleukin-1 beta (IL-1 beta), interleukin-2 (IL-2), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), interleukin-17 (IL-17), interleukin-18 (IL-18), interleukin-21 (IL-21), interleukin-23 (IL-23), interferon (IFN) alpha, IFN beta, IFN gamma, tumor necrosis alpha, GM-CSF, GCSF, or a fragment or variant thereof, or a combination of any of the aforesaid cytokines. In other embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), interleukin-23 (IL-23) or interferon gamma, or a fragment or variant thereof, or a combination of any of the aforesaid cytokines. The cytokine molecule can be a monomer or a dimer.

In embodiments, the cytokine molecule further comprises a receptor domain, e.g., a cytokine receptor domain. In one embodiment, the cytokine molecule comprises an IL-15 receptor, or a fragment thereof (e.g., an extracellular IL-15 binding domain of an IL-15 receptor alpha) as described herein. In some embodiments, the cytokine molecule is an IL-15 molecule, e.g., IL-15 or an IL-15 superagonist as described herein. As used herein, a “superagonist” form of a cytokine molecule shows increased activity, e.g., by at least 10%, 20%, 30%, compared to the naturally-occurring cytokine. An exemplary superagonist is an IL-15 SA. In some embodiments, the IL-15 SA comprises a complex of IL-15 and an IL-15 binding fragment of an IL-15 receptor, e.g., IL-15 receptor alpha or an IL-15 binding fragment thereof, e.g., as described herein.

In other embodiments, the cytokine molecule further comprises an antibody molecule, e.g., an immunoglobulin Fab or scFv fragment, a Fab fragment, a FAB₂ fragment, or an affibody fragment or derivative, e.g. a sdAb (nanobody) fragment, a heavy chain antibody fragment, e.g., an Fc region, single-domain antibody, a bi-specific or multispecific antibody). In one embodiment, the cytokine molecule further comprises an immunoglobulin Fc or a Fab.

In some embodiments, the cytokine molecule is an IL-2 molecule, e.g., IL-2 or IL-2-Fc. In other embodiments, a cytokine agonist can be used in the methods and compositions disclosed herein. In embodiments, the cytokine agonist is an agonist of a cytokine receptor, e.g., an antibody molecule (e.g., an agonistic antibody) to a cytokine receptor, that elicits at least one activity of a naturally-occurring cytokine. In embodiments, the cytokine agonist is an agonist of a cytokine receptor, e.g., an antibody molecule (e.g., an agonistic antibody) to a cytokine receptor chosen from an IL-15Ra or IL-21R.

In some embodiments, the cytokine molecule is an IL-15 molecule, e.g., a full length, a fragment or a variant of IL-15, e.g., human IL-15. In embodiments, the IL-15 molecule is a wild-type, human IL-15. In other embodiments, the IL-15 molecule is a variant of human IL-5, e.g., having one or more amino acid modifications. In some embodiments, the IL-15 molecule comprises a mutation, e.g., an N72D point mutation.

In other embodiments, the cytokine molecule further comprises a receptor domain, e.g., an extracellular domain of an IL-15R alpha, optionally, coupled to an immunoglobulin Fc or an antibody molecule. In embodiments, the cytokine molecule is an IL-15 superagonist (IL-15SA) as described in WO 2010/059253. In some embodiments, the cytokine molecule comprises IL-15 and a soluble IL-15 receptor alpha domain fused to an Fc (e.g., a sIL-15Ra-Fc fusion protein), e.g., as described in Rubinstein et al PNAS 103:24 p. 9166-9171 (2006).

The IL-15 molecule can further comprise a polypeptide, e.g., a cytokine receptor, e.g., a cytokine receptor domain, and a second, heterologous domain. In one embodiment, the heterologous domain is an immunoglobulin Fc region. In other embodiments, the heterologous domain is an antibody molecule, e.g., a Fab fragment, a Fab₂ fragment, a scFv fragment, or an affibody fragment or derivative, e.g. a sdAb (nanobody) fragment, a heavy chain antibody fragment. In some embodiments, the polypeptide also comprises a third heterologous domain. In some embodiments, the cytokine receptor domain is N-terminal of the second domain, and in other embodiments, the cytokine receptor domain is C-terminal of the second domain.

Certain cytokines and antibodies are disclosed in e.g., U.S. Publication No. 2017/0080104, U.S. Pat. No. 9,603,944, U.S. Publication No. 2014/0081012, and PCT Application No. PCT/US2017/037249 (each incorporated herein by reference in its entirety).

### Backpacks

In some embodiments, backpacks or nanoparticles can be prepared by cross-linking various therapeutic protein monomers using one or more cross-linkers disclosed herein. While the figure shows disulfide-containing linker, other biodegradable linkers disclosed herein can also be used.

In certain embodiments, the backpacks can be prepared by reacting the plurality of therapeutic protein monomers with the plurality of cross-linkers to form protein clusters having a size of, e.g., about 30 nm to 1000 nm in diameter. In some embodiments, the reaction can be performed at a temperature between about 5° C. and about 40° C. The reaction can be performed for about 1 minute to about 8 hours.

The protein clusters can be provided with a surface modification such as polycation. Certain surface modification is disclosed in U.S. Publication No. 2017/0080104 and U.S. Pat. No. 9,603,944, both incorporated herein by reference in their entirety. Examples include poly-Lysine (polyK), PEG-polyK, and poly-Arginine.

In some embodiments, the cross-linking reaction can proceed in the presence of one or more crowding agents such as polyethylene glycol (PEGs) and triglycerides. Exemplary PEGs include PEG400, PEG1000, PEG1500, PEG2000, PEG3000 and PEG4000.

Certain protein solubility aids such as glycerol, ethylene glycol and propylene glycol, Sorbitol and Mannitol can also improve the yield of backpack formation.

In certain embodiments, certain crosslinkers of the invention, due to the reaction of cationic lysine residues in the backpack, will result in a backpack having a net negative charge which will inhibit cell attachment. As such, it may be useful to first complex backpacks with a polycation via electrostatic interactions to drive cell attachment. For example, the backpacks can be coated or surface modified with a polycation such as polylysine (poly-L-lysine), polyethyleneimine, polyarginine, polyhistidine, polybrene and/or DEAE-dextran. Polycation can help the backpacks non-specifically bind or adsorb on cell membranes which are negatively charged. In some embodiments, polycation to be contained in a mixed solution may be a polymeric compound having a cationic group or a group that may become a cationic group, and an aqueous solution of a free polycation shows basic. Examples of the group that may become a cationic group include an amino group, an imino group, and the like. Examples of polycation include: polyamino acid such as polylysine, polyomithine, polyhistidine, polyarginine, polytryptophan, poly-2,4-diaminobutyric acid, poly-2,3-diaminopropionic acid, protamine, and polypeptide having at least one or more kinds of amino acid residues in a polypeptide chain selected from the group consisting of lysine, histidine, arginine, tryptophan, ornithine, 2,4-diaminobutyric acid and 2,3-diaminopropionic acid; polyamine such as polyallylamine, polyvinylamine, a copolymer of allylamine and diallylamine, and polydiallylamine; and polyimine such as polyethyleneimine.

In some embodiments, the polycation coating or surface modifying agent used to promote backpack adhesion to the cell is a cationic block copolymer of PEG-polylysine such as [methoxy-poly(ethylene glycol)n-block-poly(L-lysine hydrochloride), PEG-polylysine] (PK30). This block copolymer may contain approximately 114 PEG units (MW approximately 5000 Da) and 30 lysine units (MW approximately 4900 Da). The linear PEG polymer has a methoxy end group, the poly-lysines are in the hydrochloride salt form. PK30 is a linear amphiphilic block copolymer which has a poly(L-lysine hydrochloride) block and a non-reactive PEG block. The poly-L-lysine block provides a net cationic charge at physiological pH and renders the backpack with a net positive charge after association. PK30 Structure [Methoxy-poly(ethylene glycol)n-block-poly(L-lysine hydrochloride)] is as follows.

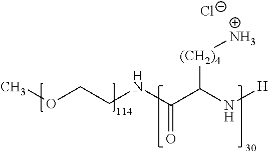

In some embodiments, the backpacks can be coated with an antibody or antigen-binding fragment thereof that bind to a receptor on the surface of an immune cell, so as to specifically target the backpacks to the immune cell. Exemplary antibodies include those disclosed herein, or fusion proteins containing the same.

In one example, “IL15-Fc” (IL15Ra-sushi-domain-Fc fusion homodimer protein with two associated IL-15 Proteins) monomers can be cross-linked and surface modified with polycation, to form IL-15 backpacks. The IL-15 backpacks can then be loaded onto immune cells such as T cells to form primed T cells.

### IFMs

In some embodiments, the cytokines or other immunomodulators can target receptors (e.g., on an immune cell) by way of an IFM, such as those disclosed in PCT Application Nos. PCT/US2018/040777, PCT/US18/40783 and PCT/US18/40786 (each incorporated herein by reference in its entirety).

In certain embodiments, the IFM can be represented with the following formula in an N to C terminal orientation: R1-(optionally L1)-R2 or R2-(optionally L1)-R1; wherein R1 comprises an immune cell targeting moiety, L1 comprises a linker (e.g., a peptide linker described herein), and R2 comprises an immune stimulating moiety, e.g., a cytokine molecule.

In some embodiments, the immune stimulating moiety, e.g., the cytokine molecule, is connected to, e.g., covalently linked to, the immune cell targeting moiety. In some embodiments, the immune stimulating moiety, e.g., the cytokine molecule, is functionally linked, e.g., covalently linked (e.g., by chemical coupling, fusion, noncovalent association or otherwise) to the immune cell targeting moiety. For example, the immune stimulating moiety can be covalently coupled indirectly, e.g., via a linker to the immune cell targeting moiety.

In embodiments, the linker is chosen from: a cleavable linker, a non-cleavable linker, a peptide linker, a flexible linker, a rigid linker, a helical linker, or a non-helical linker. In some embodiments, the linker is a peptide linker. The peptide linker can be 5-20, 8-18, 10-15, or about 8, 9, 10, 11, 12, 13, 14, or 15 amino acids long. In some embodiments, the peptide linker comprises Gly and Ser, e.g., a linker comprising the amino acid sequence (Gly₄-Ser)n, wherein n indicates the number of repeats of the motif, e.g., n=1, 2, 3, 4 or 5 (e.g., a (Gly₄Ser)₂ or a (Gly₄Ser)₃ linker).

In other embodiments, the linker is a non-peptide, chemical linker. For example, the immune stimulating moiety is covalently coupled to the immune cell targeting moiety by crosslinking. Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate).

In some embodiments, the linker can be a biodegradable or cleavable linker. A cleavable linker allows for cleavage of the IFM such that the immune stimulating moiety, e.g., the cytokine molecule, can be released from the immune targeting moiety. The cleavage of the linker may be caused by biological activation within the relevant tissue or, alternatively, by external stimuli such as, e.g., electromagnetic radiation e.g., UV-radiation.

In one embodiment, the cleavable linker is configured for cleavage exterior to a cell, e.g., to be cleaved in conditions associated with cell or tissue damage or disease. Such conditions include, for example, acidosis; the presence of intracellular enzymes (that are normally confined within cells), including necrotic conditions (e.g., cleaved by calpains or other proteases that spill out of necrotic cells); hypoxic conditions such as a reducing environment; thrombosis (e.g., a linker may be cleavable by thrombin or by another enzyme associated with the blood clotting cascade); immune system activation (e.g., a linker may be cleavable by action of an activated complement protein); or other condition associated with disease or injury.

In one embodiment, a cleavable linker may include an S—S linkage (disulfide bond), or may include a transition metal complex that falls apart when the metal is reduced. One embodiment of the S—S linker may have the following structure (as disclosed in U.S. Pat. No. 9,603,944, incorporated herein by reference in its entirety.

Another example pH sensitive linkers which are cleaved upon a change in pH, e.g., at low pH, which will facilitate hydrolysis of acid (or base) labile moieties, e.g. acid labile ester groups etc. Such conditions may be found in the extracellular environment, such as acidic conditions which may be found near cancerous cells and tissues or a reducing environment, as may be found near hypoxic or ischemic cells and tissues; by proteases or other enzymes found on the surface of cells or released near cells having a condition to be treated, such as diseased, apoptotic or necrotic cells and tissues; or by other conditions or factors. An acid-labile linker may be, for example, a cis-aconitic acid linker. Other examples of pH-sensitive linkages include acetals, ketals, activated amides such as amides of 2,3 dimethylmaleamic acid, vinyl ether, other activated ethers and esters such as enol or silyl ethers or esters, imines, iminiums, orthoesters, enamines, carbamates, hydrazones, and other linkages known in the art (see, e.g., PCT Publication No. WO 2012/155920 and Franco et al. AIMS Materials Science, 3(1): 289-323, incorporated herein by reference). The linkers disclosed in WO 2019/050977 can also be used and are incorporated herein by reference. The expression “pH sensitive” refers to the fact that the cleavable linker in question is substantially cleaved at an acidic pH (e.g., a pH below 6.0, such as in the range of 4.0-6.0).

In yet other embodiments, the immune stimulating moiety is directly covalently coupled to the immune cell targeting moiety, without a linker.

In yet other embodiments, the immune stimulating moiety and the immune cell targeting moiety of the IFM are not covalently linked, e.g., are non-covalently associated.

Exemplary formats for fusion of a cytokine molecule to an antibody molecule, e.g., an immunoglobulin moiety (Ig), for example an antibody (IgG) or antibody fragment (Fab, scFv and the like) can include a fusion to the amino-terminus (N-terminus) or carboxy-terminus (C-terminus) of the antibody molecule, typically, the C-terminus of the antibody molecule. In one embodiment, a cytokine-Ig moiety fusion molecule comprising a cytokine polypeptide, cytokine-receptor complex, or a cytokine-receptor Fc complex joined to an Ig polypeptide, a suitable junction between the cytokine polypeptide chain and an Ig polypeptide chain includes a direct polypeptide bond, a junction having a polypeptide linker between the two chains; and, a chemical linkage between the chains.

In one example, the IFM contains interleukin-12 (IL-12). Generally speaking, IL-12 is a heterodimeric cytokine composed of p35 and p40 subunits which are encoded by 2 separate genes, IL-12A and IL-12B, respectively. IL-12 is involved in the differentiation of naive T cells into Th1 cells. It is known as a T cell-stimulating factor, which can stimulate the growth and function of T cells. It stimulates the production of interferon-gamma (IFN-γ) and tumor necrosis factor-alpha (TNF-α) from T cells and natural killer (NK) cells, and reduces IL-4 mediated suppression of IFN-γ. T cells that produce IL-12 have a coreceptor, CD30, which is associated with IL-12 activity.

IL-12 plays an important role in the activities of NK cells and T lymphocytes. IL-12 mediates enhancement of the cytotoxic activity of NK cells and CD8 cytotoxic T lymphocytes. There also seems to be a link between IL-2 and the signal transduction of IL-12 in NK cells. IL-2 stimulates the expression of two IL-12 receptors, IL-12R-β1 and IL-12R-β2, maintaining the expression of a critical protein involved in IL-12 signaling in NK cells. Enhanced functional response is demonstrated by IFN-γ production and killing of target cells.

IL-12 also has anti-angiogenic activity, which means it can block the formation of new blood vessels. It does this by increasing production of interferon gamma, which in turn increases the production of a chemokine called inducible protein-10 (IP-10 or CXCL10). IP-10 then mediates this anti-angiogenic effect. Because of its ability to induce immune responses and its anti-angiogenic activity, there has been an interest in testing IL-12 as a possible anti-cancer drug. There is a link that may be useful in treatment between IL-12 and the diseases psoriasis & inflammatory bowel disease.

IL-12 binds to the IL-12 receptor, which is a heterodimeric receptor formed by IL-12R-β1 and IL-12R-β2. IL-12R-β2 is considered to play a key role in IL-12 function, since it is found on activated T cells and is stimulated by cytokines that promote Th1 cells development and inhibited by those that promote Th2 cells development. Upon binding, IL-12R-β2 becomes tyrosine phosphorylated and provides binding sites for kinases, Tyk2 and Jak2. These are important in activating critical transcription factor proteins such as STAT4 that are implicated in IL-12 signaling in T cells and NK cells.

IL-12 is a potent cytokine with the potential to reshape the anti-inflammatory environment in solid tumors. However, its clinical utility has been limited by severe toxicities both from soluble administration or from adoptively transferred T cells engineered to secrete IL-12. The tethered fusion (TF) disclosed herein enables improved control of cytokine dose and biodistribution. In in vitro model systems, the IL-12-TF cytokine provides persistent loading of IL-12 on the surface of T cells and sustained T cell activation and signaling downstream of the IL-12 receptors. In turn, this can activate innate and adaptive immunity.

The IL-12 in the tethered fusion can be in the form of a single chain containing both the IL-12A and IL-12B subunits. In some embodiments, the IL-12 can be present as a non single-chain (i.e., as a heterodimer of IL-12A and IL-12B, which is the natural form of IL-12). For example, the TF can be made by co-expression of three protein subunits (Fab heavy chain, Fab light-chain w/ IL-12A (or IL-12B), and IL-12B (or IL-12A).

In some embodiments, the TF can be IL-12-TF such as IL-12 fused to an anti-CD45 antibody, as disclosed in PCT International Publication No. WO 2019/010219, incorporated herein by reference in its entirety. In one example, “DEEP™ IL-12” (single-chain IL-12p70 fused to a humanized anti-CD45 Fab) TF can be recombinantly expressed, purified, and then tethered onto immune cells expressing CD45 such as T cells to form primed T cells.

### Compositions and Cell Therapy

In some embodiments, once prepared and purified, the backpacks can be optionally frozen until use in cell therapy. The cell therapy can be selected from, e.g., an adoptive cell therapy, CAR-T cell therapy, engineered TCR T cell therapy, a tumor infiltrating lymphocyte therapy, an antigen-trained T cell therapy, or an enriched antigen-specific T cell therapy.

In various embodiments, a cell therapy composition can be prepared by providing the protein cluster or backpack or tethered fusion composition disclosed herein, and incubating the protein cluster or backpack or tethered fusion composition with nucleated cells such as immune cells, preferably for about 30-60 minutes. The cells can be cryopreserved with backpacks until administration to a patient via, e.g., infusion.

Also disclosed herein is a cell therapy composition, comprising the protein cluster or backpack or tethered fusion composition disclosed herein, associated with a nucleated cell such as T and NK cells. Such cell therapy composition may be administered into a subject in need thereof. Upon administration, the cross-linkers in the backpacks can degrade under physiological conditions so as to release the therapeutic protein monomers from the protein cluster.

Compositions, including pharmaceutical compositions, comprising the backpacks or tethered fusions are provided herein. A composition can be formulated in pharmaceutically-acceptable amounts and in pharmaceutically-acceptable compositions. The term “pharmaceutically acceptable” means a non-toxic material that does not interfere with the effectiveness of the biological activity of the active ingredients (e.g., biologically-active proteins of the nanoparticles). Such compositions may, in some embodiments, contain salts, buffering agents, preservatives, and optionally other therapeutic agents. Pharmaceutical compositions also may contain, in some embodiments, suitable preservatives. Pharmaceutical compositions may, in some embodiments, be presented in unit dosage form and may be prepared by any of the methods well-known in the art of pharmacy. Pharmaceutical compositions suitable for parenteral administration, in some embodiments, comprise a sterile aqueous or non-aqueous preparation of the nanoparticles, which is, in some embodiments, isotonic with the blood of the recipient subject. This preparation may be formulated according to known methods. A sterile injectable preparation also may be a sterile injectable solution or suspension in a non-toxic parenterally-acceptable diluent or solvent.

The compositions disclosed herein have numerous therapeutic utilities, including, e.g., the treatment of cancers, autoimmune diseases and infectious diseases. Methods described herein include treating a cancer in a subject by using backpacks or backpacked cells as described herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is leukemia or lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the backpacks or backpacked cells are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition (or backpacks) to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the backpacks or backpacked cells are administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Checkpoint Inhibitors and Inhibitor Thereof

The ability of T cells to mediate an immune response against an antigen requires two distinct signaling interactions (Viglietta, V. et al. (2007) Neurotherapeutics 4:666-675; Korman, A. J. et al. (2007) Adv. Immunol. 90:297-339). First, an antigen that has been arrayed on the surface of antigen-presenting cells (APC) is presented to an antigen-specific naive CD4⁺ T cell. Such presentation delivers a signal via the T cell receptor (TCR) that directs the T cell to initiate an immune response specific to the presented antigen. Second, various co-stimulatory and inhibitory signals mediated through interactions between the APC and distinct T cell surface molecules trigger the activation and proliferation of the T cells and ultimately their inhibition.

The immune system is tightly controlled by a network of costimulatory and co-inhibitory ligands and receptors. These molecules provide the second signal for T cell activation and provide a balanced network of positive and negative signals to maximize immune responses against infection, while limiting immunity to self (Wang, L. et al. (Epub Mar. 7, 2011) J. Exp. Med. 208(3):577-92; Lepenies, B. et al. (2008) Endocrine, Metabolic & Immune Disorders—Drug Targets 8:279-288). Examples of costimulatory signals include the binding between the B7.1 (CD80) and B7.2 (CD86) ligands of the APC and the CD28 and CTLA-4 receptors of the CD4 T-lymphocyte (Sharpe, A. H. et al. (2002) Nature Rev. Immunol. 2: 116-126; Lindley, P. S. et al. (2009) Immunol. Rev. 229:307-321). Binding of B7.1 or B7.2 to CD28 stimulates T cell activation, whereas binding of B7.1 or B7.2 to CTLA-4 inhibits such activation (Dong, C. et al. (2003) Immunolog. Res. 28(0:39-48; Greenwald, R. J. et al. (2005) Ann. Rev. Immunol. 23:515-548). CD28 is constitutively expressed on the surface of T cells (Gross, J., et al. (1992) J. Immunol. 149:380-388), whereas CTLA-4 expression is rapidly up-regulated following T-cell activation (Linsley, P. et al. (1996) Immunity 4:535-543).

Other ligands of the CD28 receptor include a group of related B7 molecules, also known as the “B7 Superfamily” (Coyle, A. J. et al. (2001) Nature Immunol. 2(3):203-209; Sharpe, A. H. et al. (2002) Nature Rev. Immunol. 2: 116-126; Collins, M. et al. (2005) Genome Biol. 6:223.1-223.7; Korman, A. J. et al. (2007) Adv. Immunol. 90:297-339). Several members of the B7 Superfamily are known, including B7.1 (CD80), B7.2 (CD86), the inducible co-stimulator ligand (ICOS-L), the programmed death-1 ligand (PD-L1; B7-H1), the programmed death-2 ligand (PD-L2; B7-DC), B7-H3, B7-H4 and B7-H6 (Collins, M. et al. (2005) Genome Biol. 6:223.1-223.7).

The Programmed Death 1 (PD-1) protein is an inhibitory member of the extended CD28/CTLA-4 family of T cell regulators (Okazaki et al. (2002) Curr Opin Immunol 14: 391779-82; Bennett et al. (2003) J. Immunol. 170:711-8). Other members of the CD28 family include CD28, CTLA-4, ICOS and BTLA. Two cell surface glycoprotein ligands for PD-1 have been identified, Program Death Ligand 1 (PD-L1) and Program Death Ligand 2 (PD-L2). PD-L1 and PD-L2 have been shown to downregulate T cell activation and cytokine secretion upon binding to PD-1 (Freeman et al. (2000) J Exp Med 192: 1027-34; Latchman et al. (2001) Nat Immunol 2:261-8; Carter et al. (2002) Eur J Immunol 32:634-43; Ohigashi et al. (2005) Clin Cancer Res 11:2947-53).

PD-L1 (also known as cluster of differentiation 274 (CD274) or B7 homolog 1 (B7-H1)) is a 40 kDa type 1 transmembrane protein. PD-L1 binds to its receptor, PD-1, found on activated T cells, B cells, and myeloid cells, to modulate activation or inhibition. Both PD-L1 and PD-L2 are B7 homologs that bind to PD-1, but do not bind to CD28 or CTLA-4 (Blank et al. (2005) Cancer Immunol Immunother. 54:307-14). Binding of PD-L1 with its receptor PD-1 on T cells delivers a signal that inhibits TCR-mediated activation of IL-2 production and T cell proliferation. The mechanism involves inhibition of ZAP70 phosphorylation and its association with CD3C (Sheppard et al. (2004) FEB S Lett. 574:37-41). PD-1 signaling attenuates PKC-Θ activation loop phosphorylation resulting from TCR signaling, necessary for the activation of transcription factors NF-κB and AP-1, and for production of IL-2. PD-L1 also binds to the costimulatory molecule CD80 (B7-1), but not CD86 (B7-2) (Butte et al. (2008) Mol Immunol. 45:3567-72).

Expression of PD-L1 on the cell surface has been shown to be unregulated through IFN-γ stimulation. PD-L1 expression has been found in many cancers, including human lung, ovarian and colon carcinoma and various myelomas, and is often associated with poor prognosis (Iwai et al. (2002) PNAS 99:12293-7; Ohigashi et al. (2005) Clin Cancer Res 11:2947-53; Okazaki et al. (2007) Intern. Immun. 19:813-24; Thompson et al. (2006) Cancer Res. 66:3381-5). PD-L1 has been suggested to play a role in tumor immunity by increasing apoptosis of antigen-specific T-cell clones (Dong et al. (2002) Nat Med 8:793-800). It has also been suggested that PD-L1 might be involved in intestinal mucosal inflammation and inhibition of PD-L1 suppresses wasting disease associated with colitis (Kanai et al. (2003) J Immunol 171:4156-63).

In some embodiments, the composition or cancer immunotherapy disclosed herein comprises an anti-PD-L1 antibody or antigen binding fragment thereof. For instance, Atezolizumab and other anti-PD-L1 antibodies are disclosed in U.S. Pat. No. 8,217,149, incorporated by reference in its entirety. Avelumab and other anti-PD-L1 antibodies are disclosed in WO 2013/079174, incorporated by reference in its entirety. Durvalumab and other anti-PD-L1 antibodies are disclosed in U.S. Pat. No. 8,779,108, incorporated by reference in its entirety. BMS-936559 and other anti-PD-L1 antibodies are disclosed in U.S. Pat. No. 7,943,743 and WO 2015/081158, incorporated by reference in their entireties. Further anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entireties.

### EXAMPLES

### Example 1: Backpacking of Immune Cells

Purpose: Human cells (e.g., Tcells, CAR-T, NK cells, other immune cells) can be labeled with 5 concentrations of IL-15 backpack in HBSS at a cell concentration of 50M/mL. After labeling the cells can be tested for:
a. Viability via 7-AAD staining measured by FACS
b. Expansion in culture via counting beads measured by FACS
c. Backpack surface labeling via antibodies against IL-15 and human anti-IgG

Note that while this Example focuses on IL-15, one of ordinary skill will understand that the same experimental procedure are applicable to other cytokines and/or IFMs.

### Thawing of IL-15 Backpacks:

Backpacks should be stored at −80° C. before use. Thawed backpacks can be re-frozen and re-used up to 3 or more freeze/thaw cycles.

Take backpack aliquots out of the freezer, and thaw them on ice.
1. After BP solution is thawed, let it warm up to room temperature 15 min prior to cell labeling experiments
2. Adjust the BP stock solution to a final working solution of 3 mg/mL with HBSS

| | [BP] stock | [BP] working | BP stock | HBBS | Total vol of BP |
|---|---|---|---|---|---|
| | (mg/mL) | conc. (mg/mL) | needed (uL) | needed (uL) | working sol (uL) |
| CYT15 | 4.2 | 3 | 100 | 40 | 140 |

Backpack Dilution and Cell Labeling:

7 reactions total: one PBS only control, one soluble IL-15 constant control added to cells after plating), and five backpack samples to be done in triplicate (21 samples total). The backpack samples are:
a. BP-Dose1: 3 mg/mL
b. BP-Dose2: 1.5 mg/mL
c. BP-Dose3: 0.75 mg/mL
d. BP-Dose 4: 0.375 mg/mL
e. BP-Dose 5: 0.1875 mg/mL
1. Make serial dilutions of backpacks in round-bottom 96-well plate:

| | | | Volume | Volume of | |
|---|---|---|---|---|---|
| Backpack | | | HBSS | Previous Dose | |
| Dose | Concentration | | (>3x) | (>3x) | |
| Dose 1 | 3 | mg/mL | NA | 60 | ul stock |
| Dose 2 | 1.5 | mg/mL | 60 ul | 60 | ul of stock |
| Dose 3 | 0.75 | mg/mL | 60 ul | 60 | ul of Dose 2 |
| Dose 4 | 0.375 | mg/mL | 60 ul | 60 | ul of Dose 3 |
| Dose 5 | 0.1875 | mg/mL | 60 ul | 60 | ul Dose 4 |

| | | | |
|---|---|---|---|
| PBS control | 0 | 60 ul | NA |
| Soluble IL-15 | 0 | 60 ul | NA |
| control | | | |

2. Distribute 10 ul of diluted backpack from each well above into three wells in a round-bottom 96-well plate (backpacking in triplicate)-21 wells total.

### Cell Washing and Backpacking:

The buffers, PBS, and media used in the steps below should be pre-warmed to 37° C.
1. Collect 30×10⁶ cells from culture and pellet them at 500 g for 5 minutes
2. Remove cell supernatant by aspiration.
3. Wash cells by resuspending the pellet in 10 mL pre-warmed (37° C.) HBSS buffer and count by Cellometer (with AOPI dye) or Trypan Blue.
4. Centrifuge at 500 g for 5 min
5. Aspirate supernatant and suspend cell pellet in pre-warmed (37° C.) HBSS to a concentration of 100×10⁶/mL cells (approximately 300 ul of buffer)
6. Pipet 10 ul of cells into each well with backpacks or HBSS and gently mix them by pipetting.

| | | | | Final BP | | |
|---|---|---|---|---|---|---|
| | | Cell Vol | BP Vol | Conc. | HBSS | Final vol. |
| Samples | Cell # | (ul) | (ul) | (mg/mL) | (uL) | (uL) |
| Dose 1 | 1 × 10⁶ | 10 | 10 | 1.5 | 0 | 20 |
| Dose 2 | 1 × 10⁶ | 10 | 10 | 0.75 | 0 | 20 |
| Dose 3 | 1 × 10⁶ | 10 | 10 | 0.375 | 0 | 20 |
| Dose 4 | 1 × 10⁶ | 10 | 10 | 0.188 | 0 | 20 |
| Dose 5 | 1 × 10⁶ | 10 | 10 | 0.094 | 0 | 20 |
| PBS | 1 × 10⁶ | 10 | 0 | 0 | 10 | 20 |
| Soluble | 1 × 10⁶ | 10 | 0 | 0 | 10 | 20 |
| IL-15 | | | | | | |

7. Cover plate with a microfilm to prevent evaporation, and incubate in the cell culture incubator (typically 37° C. with 5% CO₂ or what is best for culture).
8. Incubate cells for one hour at 37° C.
9. Add 180 uL pre-warmed complete cell media (with serum) to each well.
10. Pellet cells at 500 g for 5 min, and aspirate media with a multiwell manifold
11. Wash cells two more times with 200 uL full media, pellet cells, and aspirate supernatant as in steps 9 and 10.
12. After the third wash, resuspend cells from each sample in 200 uL full media. The cells should be at ˜5×10⁶ cells/mL density and need to be further diluted by 1:10 during plating.
13. Dilute cells 1:10 by transferring 20 ul of cell suspension from the 96-well U bottom to 96-well flat-bottom tissue culture plate and then adding 180 ul cell media (without added cytokines) to achieve a final plating density of 5×10⁵ cells/mL.
14. Repeat step 13 three additional times in three separate 96-well flat bottom plates (4 plates total: Day0, Day1, Day3, DayX for splitting for future time points)
15. Add soluble IL-15 monomers to soluble IL-15 constant control wells of each plate.

### Cell Count and Viability Test:

Cells are counted using 7-AAD and CountBright counting beads on flow cytometer.
1. At each time point, take a 96-well flat bottom plate out of the incubator, resuspend cells in media by pipetting up and down
2. Transfer 20 uL of cell solution to a 96-well V-bottom plate
3. To each well, add 20 uL of “CountBright Bead solution”.
   CountBright Bead Solution contains (volumes for labeling 1 well is listed below):
   a. 19.6 ul CountBright bead stock
   b. 0.4 ul of 100×7AAD (7-AAD, LifeTech, A1310, 10 ug/mL is 100×)
4. Repeat these steps on days 1, 3 and X after culturing to assess viability and expansion.

### BP Loading Efficiency Test by Surface Staining:

Analyze surface levels of IL-15 backpacks by flow cytometry on Days 0 and Day 1 using anti-IL-15 and anti-human IgG antibodies.
1. Take a 96-well flat bottom plate out of the incubator, resuspend cells in media by pipetting up and down
2. Transfer 100 uL of cell solution to a new V-bottom 96-well plate (this should contain ˜50,000 cells)
3. Pellet cells (500 g for 5 min) and aspirate supernatant
4. Resuspend cells in 40 uL “Antibody Cell Surface Staining Solution”
   Antibody Staining Solution (volumes for labeling 1 well is listed below):
      a. 0.4 uL of Mouse anti-human IgG BV421—Biolegend cat. no. 409318, 1:100 dilution
      b. 0.4 uL of Anti-IL-15 PE: R&D Systems cat. no. IC2471IP, 1:100 dilution
      c. 0.4 uL of 100×7AAD (7-AAD, LifeTech, A1310, 10 ug/mL is 100×)
      d. 38.8 uL of MACS buffer
5. Incubate cells for 10 min at room temperature
6. Add 160 uL of cold MACS buffer to each well, pellet cells at 500 g for 5 min, aspirate.
7. Wash cells one additional time with 200 uL cold MACS buffer
8. Resuspend in 30 uL per well of MACS buffer and analyze on flow cytometer (HTS mode)

### Reagents Used:

Hank's Balanced Salt Solution (HBSS, Gibco, with calcium and magnesium, cat #14025-092)
Phosphate-Buffered Saline (PBS. Gibco, no calcium, no magnesium, cat #10010-023)
Round-bottom 96-well plate (Granier-Bio, clear, sterile, polypropylene plates, cat #650261)
v-bottom 96-well plate (optional but recommended): Costar 3894
Flat-bottom 96-well plate (FisherSci, cat #353072)

### Counting Beads (LifeTech, CountBright Absolute Counting Beads, cat #C36950)

7-aminoactinomycin-D (7-AAD, LifeTech, cat #A1310)
Human IL-15 PE-Conjugated. Antibody (R&D Systems, cat #IC2471P)
Mouse anti-Human IgG, BV421 (Biolegend, cat #409318)
Alternative Ab: Mouse anti-Human NG, APC (Biolegend, cat #409306)
Alternative. Ab: Donkey anti-Human IgG (H+L), DyLight 650 (ThermoFisher, cat #SA5-10129)
MACS Buffer (optional):
EDTA: LifeTechnologies, 15575-038
Phosphate-Buffered Saline, pH 7.4 (same as above)
Bovine Serum Albumin (BSA): AmericanBio, Inc. cat #AB01243-00050

### Example 2: DEEP™ IL-15 Primed T Cells Synergize with PD-L1 Blockade to Overcome Resistance to Checkpoint Immunotherapy

Introduction: Interleukin-15 (IL-15) activates and expands both CD8⁺ T cells and NK cells but not immunosuppressive T_{reg} cells. Thus, IL-15 is an attractive asset for cancer immunotherapy, but its systemic administration is limited by immune activation and toxicities. To limit IL-15 systemic exposure, we have developed DEEP™ IL-15, a multimer of chemically crosslinked IL-15/IL-15 Rα/Fc heterodimers (IL15-Fc). DEEP™ IL-15 is loaded onto tumor reactive T cells prior to adoptive cell transfer (ACT). This novel therapeutic approach enables DEEP™ IL-15 loading into cells at concentrations unachievable with systemic IL15-Fc, causes autocrine T cell activation and expansion, yet limits systemic exposure and associated toxicities. The anti-tumor activity of T cell therapies has been limited by insufficient T cell expansion and by checkpoint immunosuppression. Here, we combined DEEP™ IL-15 primed T cells with PD-L1 blockade to overcome these limitations.

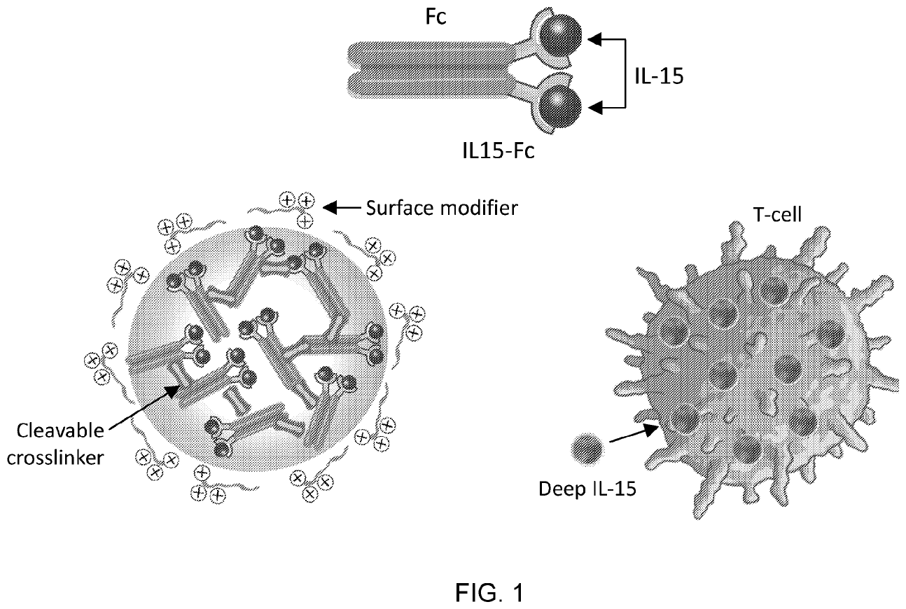

Specifically, as illustrated in FIG. 1, DEEP™ IL-15 (or DP-15) refers to a multimer of human IL-15 receptor α-sushi-domain-Fc fusion homodimers with two associated IL-15 molecules (IL15-Fc), connected by a cleavable crosslinker (see, e.g., PCT Application No. PCT/US2018/049594, incorporated herein by reference), and non-covalently coated with a polyethylene glycol (PEG)-polylysine₃₀ block copolymer (PK30). More specifically, DEEP™ IL-15 is a multimer of human IL15-Fc monomers, connected by a hydrolysable crosslinker (CL17) and non-covalently coated with a polyethylene glycol (PEG)-polylysine₃₀ block copolymer (PK30). IL15-Fc monomers consist of two subunits, each consisting of an effector attenuated IgG2 Fc variant fused with an IL-15 receptor α-sushi-domain noncovalently bound to a molecule of IL-15. DEEP™ IL-15 Primed T cells are generated via a loading process in which target cells are co-incubated with DEEP™ IL-15 at high concentrations. Through this process, DEEP™ IL-15 becomes associated with the cell via electrostatic interactions and is internalized to create intracellular reservoirs of DEEP™ IL-15. From these reservoirs, DEEP™ IL-15 slowly releases bioactive IL15-Fc by hydrolysis of the crosslinker. This extended release of IL15-Fc promotes proliferation and survival of DEEP™ IL-15 Primed T cells, providing a targeted, controllable and time-dependent immune stimulus.

The objective of this study was to evaluate anti-tumor activity of DEEP™ IL-15 Primed PMEL cells in combination with anti PD-L1, and evaluate contribution of cyclophosphamide (CPX) to anti-tumor activity of DEEP™ IL-15 Primed PMEL cells alone or in combination with anti PD-L1.

### Materials and Methods

### DEEP™ IL-15 Synthesis

DEEP™ IL-15 was synthesized by incubation of IL15-Fc with a crosslinking reagent. PMEL CD8+ T cells (PMEL) were isolated from B6. Cg-Thy1^{a}/Cy Tg(TcraTcrb)8Rest/J mice, activated, expanded, and loaded with DEEP™ IL-15 to generate DEEP™ IL-15 Primed PMEL (DEEP™-15 PMEL).

### Study Animals

B6D2F1 female mice (˜7 weeks old) were purchased from Charles River Laboratories (Wilmington, Mass.). Body weights ranged from 15.5 to 21.3 g at the beginning of the study. The care and treatment of experimental animals were in accordance with Institutional Animal Care and Use Committee (IACUC) guidelines.

### B16-F10 Tumor Establishment and Tumor and Body Weight Measurements

B16-F10 melanoma cells (1×10⁶) were injected subcutaneously into the shaved right flank of female B6D2F1 mice on study Day −5. Tumor dimensions (length [L] and width [W], defined in the list of abbreviations) were measured with calipers 3 times per week. Tumor volumes were calculated using the formula (W²×L)/2. For enrollment, mice were randomized based on tumor volume (average 33.6 mm³; range 18 to 63 mm³). Animals were monitored individually for tumor growth until Day 76, and each animal was euthanized when its tumor volume reached or exceeded 1500 mm³.

Treatment may cause a partial regression (PR) or a complete regression (CR) of the tumor in an animal. In a PR response, the tumor volume is 50% of its Day 1 volume for three consecutive measurements during the course of the study, and 13.5 mm³ for one or more of these three measurements. In a CR response, the tumor volume is 13.5 mm³ for three consecutive measurements during the course of the study. Animals were scored only once during the study for a PR or CR event, and only as CR if both PR and CR criteria were satisfied. Any animal with a CR response on the last day of the study was additionally classified as a tumor-free survivor (TFS).

Animals were weighed daily on Days 1-5, then twice per week until the completion of the study. The mice were observed frequently for overt signs of any adverse, treatment-related (TR) side effects, and clinical signs were recorded when observed. Individual body weight (BW) was monitored as per protocol, and any animal with a weight loss exceeding 30% for one measurement or exceeding 25% for three consecutive measurements was euthanized as a TR death. Group mean BW loss was also monitored according to Charles River Discovery Services protocol. Acceptable toxicity was defined as a group mean BW loss of less than 20% during the study and no more than 10% TR deaths.

### Lymphodepletion

All mice were injected with CPX at 4 mg/mouse on D1 to deplete endogenous immune cells.

### Isolation and Expansion of PMEL Cells

PMEL T cells were isolated from the spleens and lymph nodes (inguinal, axillary and cervical) of 30 female PMEL mice (Jackson Laboratories, Bar Harbor, Me.). Spleens and lymph nodes were processed with a GentleMACS Octo Dissociator (Miltenyi Biotech, Auburn, Calif.) and passed through a 40 μm strainer. Cells were washed by centrifugation and CD8a⁺ cells purified using an IMACS naïve CD8a⁺ isolation kit (Miltenyi Biotech) and a MultiMACS cell 24 block (Miltenyi Biotech) and separator (Miltenyi Biotech) following the manufacturer's protocol. The purity of CD8a⁺ cells was confirmed by flow cytometry.

A total of approximately 533×10⁶ PMEL T cells were isolated and frozen in CryoStor®CS10 media (STEMCELL Technologies) at a density of 10×10⁶/mL/vial. Approximately 200×10⁶ cells (20 vials) were shipped to Charles River Discovery Services, 3300 Gateway Centre Blvd, Morrisville, N.C. 27560 to be used in the experiment described here. In vitro expansion and loading of the PMEL T cells with DEEP™ IL-15 were performed at Charles River Discovery Services as part of study InVitro-e480. Frozen CD8+ T cells were thawed (D0), resuspended at 1.0×10⁶/mL in Roswell Park Memorial Institute 1640 media (RPMI-1640) containing 10% Fetal Calf Serum (FCS), Penicillin/Streptomycin (Pen/Strep) (1%), L-glutamine (1%), Insulin/Transferrin/Selenium (ITS, 1%) and (3-mercaptoethanol (BME, 50 μM), and plated into 6-well tissue culture plates (5×10⁶ cells/well) coated with αCD3 and αCD28 antibodies. Cells were incubated for 24 hr at 37° C. and 5% CO₂. Mouse IL-2 (20 ng/mL) and mouse IL-7 (0.5 ng/mL) were added 24 hr post plating (D1). On D2 and D3, the cells were counted and diluted to a concentration of 0.2×10⁶ cells/mL with fresh media containing mouse IL-21 (25 ng/mL). The cells were collected on D4 and resuspended at 100×10⁶ PMEL T cells/mL in vehicle buffer.

### Preparation of DEEP™ IL-15 Primed PMEL T Cells

PMEL T cells (100×10⁶ cells/mL) were mixed with an equal volume of DEEP™ IL-15 (1.5 mg/mL) and incubated with rotation for 1 hr at 37° C. to generate DEEP™ IL-15 Primed PMEL T cells. DEEP™ IL-15 Primed PMEL T cells were washed (3×, first with medium and then twice with HBSS) by centrifugation (500 g) and counted. The cells were then resuspended at 25×10⁶ cells/mL, and mice in Groups 5 and 7 received 200 μL of the cell suspension (5×10⁶ cells) IV into the tail vein.

### αPD-L1 Antibody

Anti-PD-L1 antibody was purchased from BioXcell (clone 10F.9G2) and was stored at 4° C. protected from the light until use.

### Blood Collection

In-life blood samples (˜0.1 mL) were collected from all mice in all groups by submandibular bleeds on experiment days D3, D6 and D20 (1, 4 and 18 days post ACT, respectively). Samples from mice 1-5 were processed for serum preparation without anticoagulant and frozen. Samples from mice 6-10 were collected in EDTA-coated tubes and processed for flow cytometry analysis (D3 and D6 samples only, not shown).

Evaluation of the Combination of DEEP™ IL-15 Primed PMEL Cells with Anti-PD-L1 in B16-F10 Melanoma Tumor-Bearing Mice

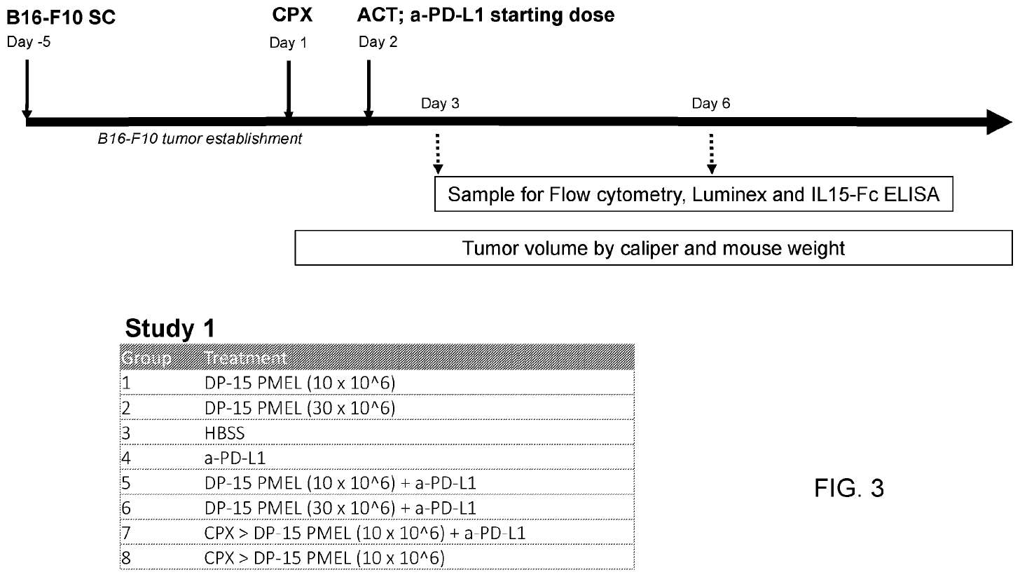

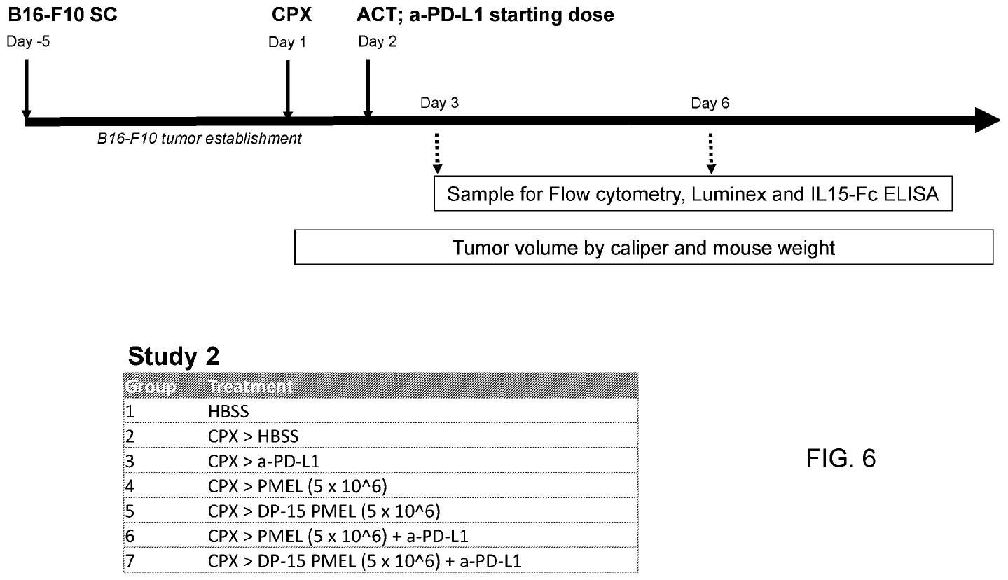

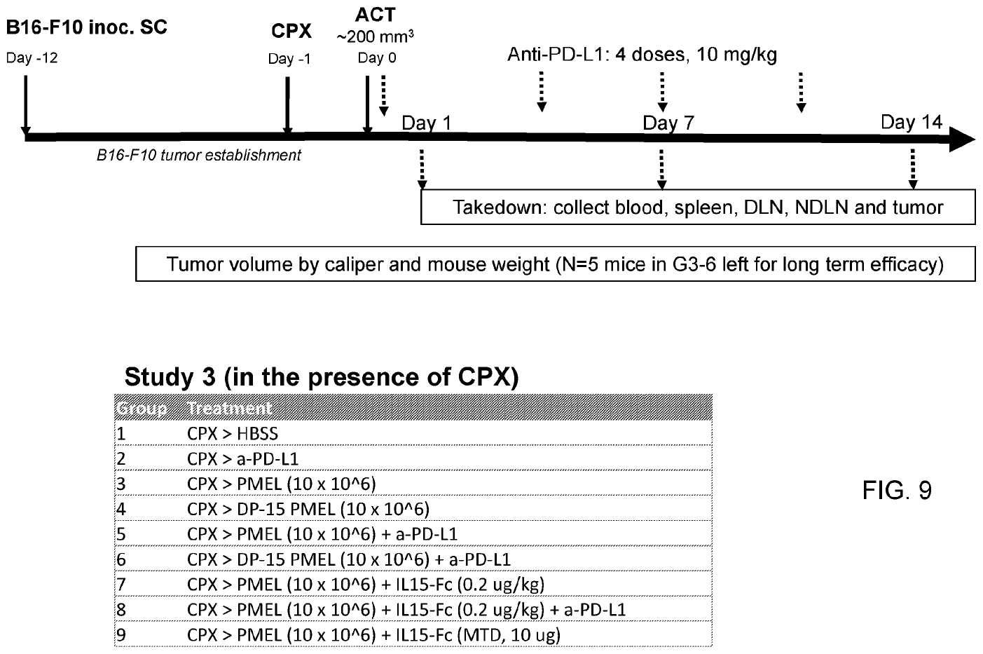

Experiments were designed to evaluate the anti-tumor activity of PMEL cells loaded with DEEP™ IL-15 in combination with checkpoint blockade (anti-PD-L1). Experimental timelines are shown in FIGS. 3, 6, and 9, for study 1, 2 and 3, respectively. Detailed methods for study 2 is provided herein, which are similar to those of study 1 and 3 unless otherwise noted in FIGS. 3 and 9. Specifically, for study 1, cells injected were 10×10⁶ not 5×10⁶ (study 2) and there was no PD-L1 alone, and the control group was saline (this group did not receive CPX). In study 2 all groups received CPX. In study 3, larger tumors (˜200-300 mm³) were used than study 1 and 2 (closer to 30-50 mm³).

### Fc-IL-15 ELISA

An Fc-IL-15 Enzyme-Linked Immunosorbent Assay (ELISA) was used to determine the IL15-Fc concentration in the serum samples collected on D3, D6 and D18. ELISA plates were coated overnight at 4° C. with Goat Anti-human IgG Fc Capture Antibody (Southern Biotech; 0.5 μg/mL in Phosphate Buffered Saline, PBS). Plates were then washed and blocked with reagent diluent (1% Bovine Serum Albumin, BSA, in PBS) for at least 2 hr at 30° C. Plates were washed, samples (diluted 1:20 in reagent diluent) and IL15-Fc standards (in duplicates, 31 to 2000 pg/mL, in reagent diluent) were added to the wells, and plates were incubated for 1 hr at 37° C. Plates were next washed followed by addition of biotin-anti-IL-15 detection antibody (R&D Systems; 0.125 μg/mL in reagent diluent) and incubation for 1 hr at 37° C. Plates were then washed, incubated with Streptavidin-HRP (Southern Biotech; 1/6000 in reagent diluent) for 20 min at 37° C., washed again followed by addition of 3,3′,5,5′-Tetramethylbenzidine (TMB) substrate solution (Surmodics), and incubated for 20 min at room temperature in the dark until the reaction was stopped with 1N HCl (J. T. Baker). Plates were then read on a microplate reader (450 nm).

The lower limit of quantitation (LLOQ) in blood was 0.31 ng/ml for the 1:20 dilution.

### Flow Cytometry

Mice were euthanized 7 days post ACT for intratumoral PMEL T cell profiling by Flow Cytometry. Tumors were dissociated single cell suspension, and washed with Staining buffer (0.5% BSA, 2 mM EDTA in PBS). Cell pellets were resuspended in a master mix containing the staining antibodies listed below (diluted 1:100 in Staining buffer) and Counting beads (Thermo Fisher, Waltham, Mass.), and incubated for 30 min at room temperature and protected from light. For intracellular staining, the cells were washed 3× in Staining buffer, resuspended in Fixation/Permeabilization Solution (Thermo Fisher Scientific, Waltham, Mass.), and incubated overnight (4° C.). On the next day, samples were centrifuged, washed 3× in Permeabilization buffer, incubated for 30 min with antibodies against the intracellular Ki67 marker at room temperature protected from light, and washed once in Permeabilization buffer followed by one wash in Staining buffer. The cells were then resuspended in Staining Buffer for FACS analysis.

Flow cytometry data was collected on a FACSCelesta (Becton-Dickinson, Franklin Lakes, N.J.) and analyzed in FlowJo v10.

| Species | Cell | | | |
|---|---|---|---|---|
| reactivity | marker | Fluorophore | Manufacturer | Cat # |
| Mouse | CD45 | BV510 | Biolegend | 103138 |
| Mouse | CD90.1 | AF700 | Biolegend | 202528 |
| Mouse | CD8a | APC-Cy7 | Biolegend | 100714 |
| Mouse | CD4 | BV711 | Biolegend | 100447 |
| Mouse | PD1 | BV785 | Biolegend | 135225 |
| Mouse | CD39 | PE-Cy7 | Biolegend | 143806 |
| Mouse | Tlm3 | APC | Biolegend | 134008 |
| Mouse | Lag3 | PerCP-Cy5.5 | Biogened | 125212 |
| Mouse | CICas3 | 488 | R&D | IC835G-100 |
| Mouse | IFN-g | PE | Biolegend | 505808 |
| Mouse | Fc-block | N/A | Biolegend | 101320 |
| N/A | Zombie | BV421 | Biolegend | 423114 |
| | Violet | | | |

### Results

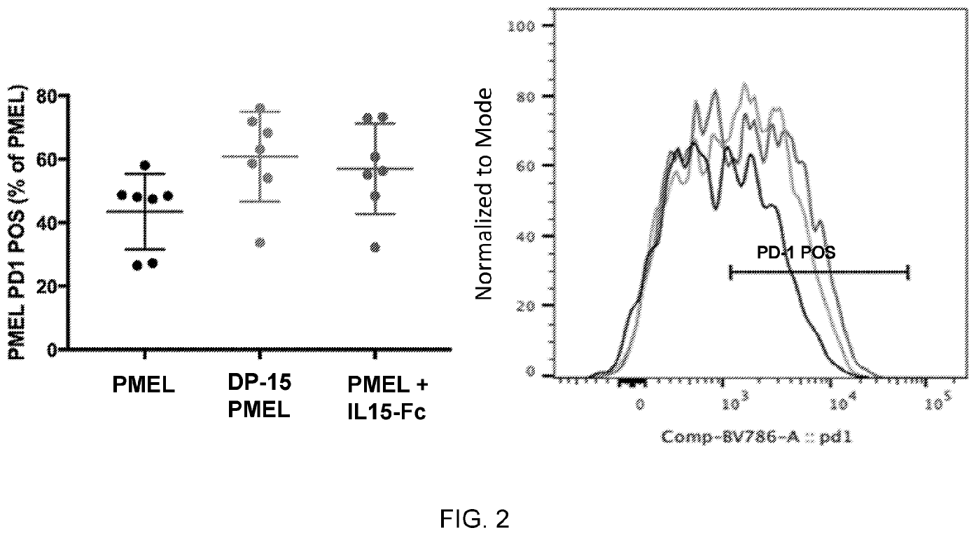

As shown in FIG. 2, IL-15 increases PD-1 expression on PMEL T cells. Then three DEEP™ IL-15 primed PMEL and anti-PD-L1 combination studies (Study 1, 2 and 3) were designed and conducted as illustrated in FIGS. 3, 6 and 9, respectively.

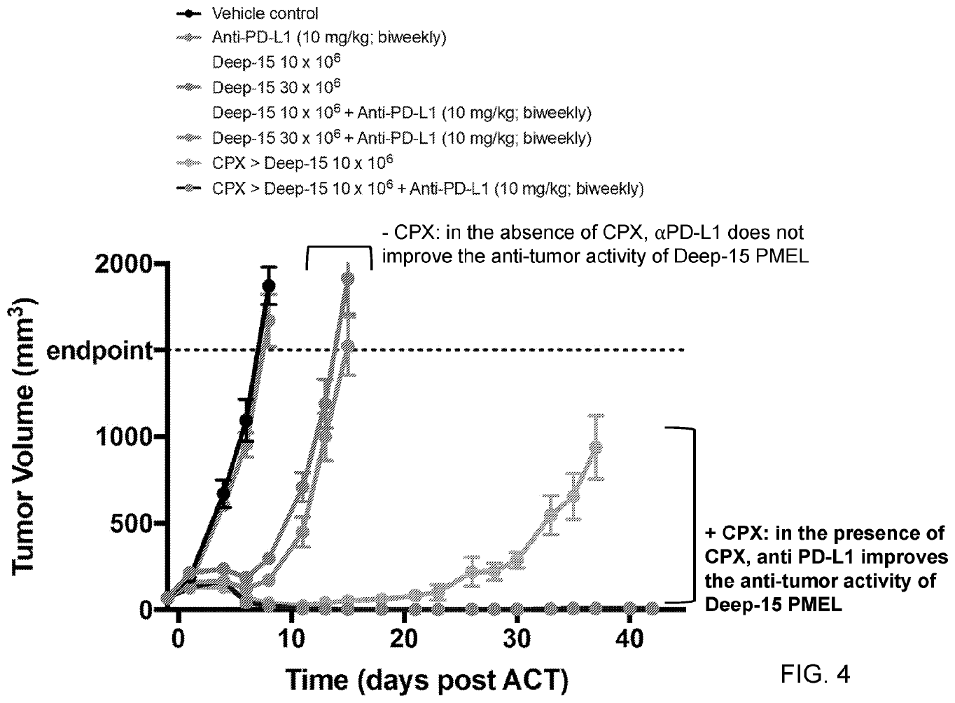

FIG. 4: Study 1 shows that αPD-L1 improves the anti-tumor activity of DEEP™-15 Primed PMEL T cells in the presence of lymphodepletion (CPX). In the absence of CPX, αPD-L1 does not improve the anti-tumor activity of DEEP™-15 PMEL. In the presence of CPX, anti PD-L1 improves the anti-tumor activity of DEEP™-15 PMEL.

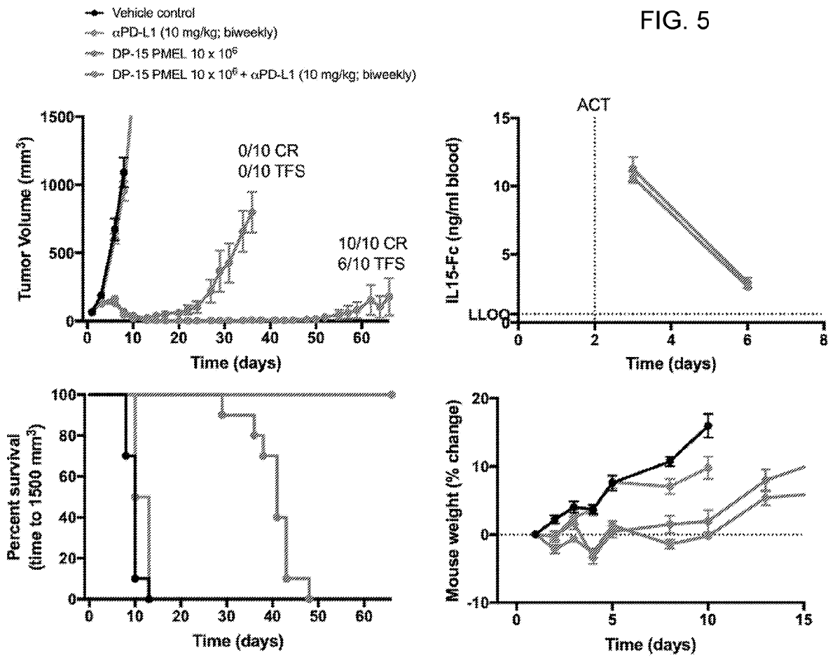

FIG. 5: Study 1 shows that DEEP™ IL-15 Primed PMEL T cells in combination with αPD-L1 increase anti-tumor activity, increase tumor free survivors (TFS), result in no changes in exposure to IL15-Fc, and are well tolerated, with no changes in body weight.

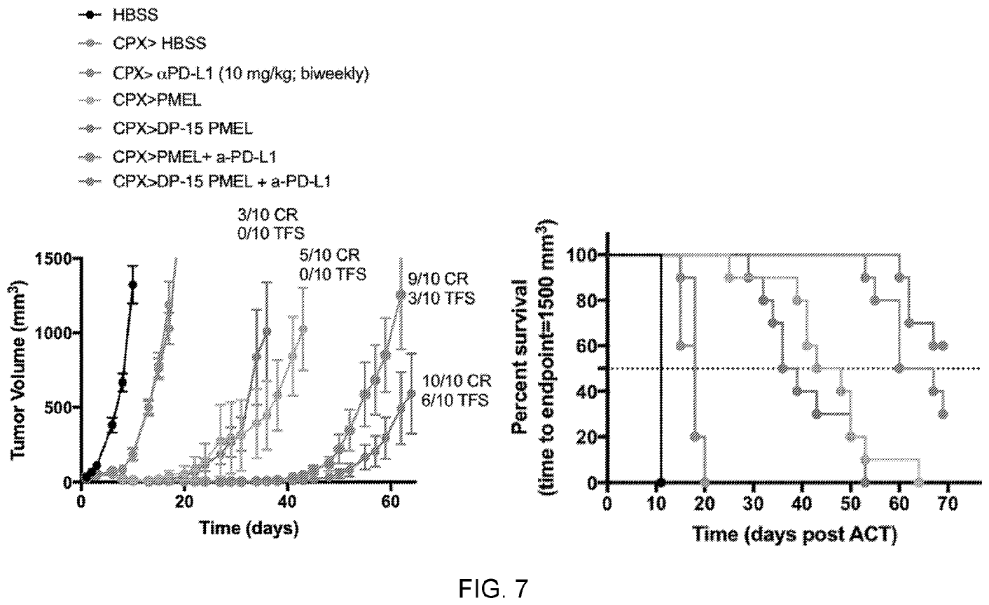

FIG. 7: Study 2 shows that DP-15 PMEL+a-PD-L1 shows improved anti-tumor activity and survival compared to PMEL+αPD-L1.

Specifically, for study 2, mice were treated with CPX (4 mg/mouse) when tumors reached an average volume of 33.6 mm³ (D1). The following day (D2), mice were dosed with either vehicle (HBSS), αPD-L1 (10 mg/kg, continued twice weekly for the entire study), DEEP™ IL-15 Primed PMEL T cells (5×10⁶, one single IV injection) alone or in combination with αPD-L1 (10 mg/kg, continued twice weekly for the entire study).

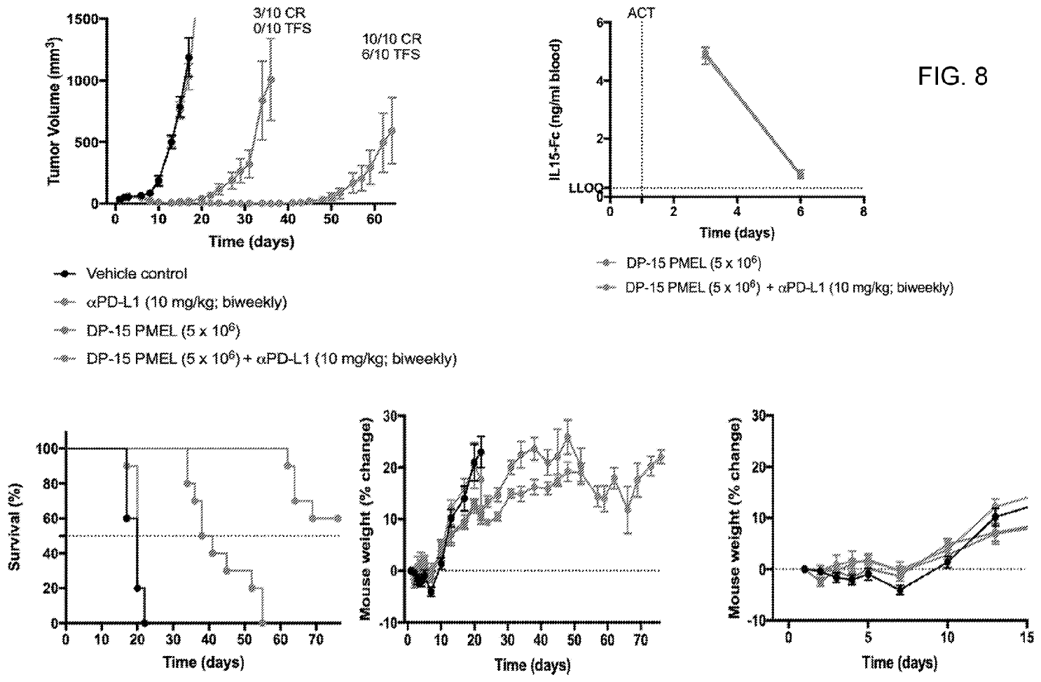

Treatment with DEEP™ IL-15 Primed PMEL T cells alone resulted in a statistically significant tumor growth inhibition compared to treatment with either vehicle or αPD-L1 alone, which had no anti-tumor activity compared to vehicle control (FIG. 8). Treatment with DEEP™ IL-15 Primed PMEL T cells alone also elicited 3/10 complete regressions (CR, defined as a tumor volume 13.5 mm³ for three consecutive measurements). The anti-tumor activity of DEEP™ IL-15 Primed PMEL T cells was further improved by the addition of αPD-L1, and 100% (10/10) of animals in the combination group showed CRs. At the end of the study (D76, 74 days post ACT), 60% (6/10) of the combination-treated mice still showed CRs and were classified as tumor-free survivors (TFS) (FIG. 8). No TFS were observed in the DEEP′ IL-15 Primed PMEL T cell group without concomitant checkpoint blockade.

All vehicle and αPD-L1 treated mice reached the endpoint (defined by a tumor volume 1500 mm³) by day 20 post ACT (D22) (FIG. 8). The median survival was 20 days for both of these two controls groups. Mice treated with DEEP′ IL-15 Primed PMEL T cells were all euthanized between days 32 and 53 post ACT (D34 to D55, respectively), with a median survival of 39.5 days. In contrast, 6/10 mice treated with DEEP™ IL-15 Primed PMEL T cells in combination with αPD-L1 were still on-study at the end of the experiment (74 days post ACT, or D76), and they were all tumor-free (TFS). This demonstrates further improvement of anti-tumor activity by the addition of αPD-L1 treatment to Deep IL-15 Primed PMEL T cell therapy.

Body weights were monitored daily at the beginning of the study (D1-5), then twice per week until the completion of the study. All treatments were well tolerated throughout the course of the experiment (FIG. 8), including the first two weeks (FIG. 8). A transient, non-toxicologically relevant mild reduction in body weight compared to the initial (D1) weight was observed on D2 in the Deep IL-15 Primed PMEL T cell group (−2.5%). Body weights had recovered by D3. Compared to the vehicle and αPD-L1 treated mice, a reduced weight gain was observed with the Deep IL-15 Primed PMEL T cells alone or in combination with αPD-L1. This observation was likely attributable to reduced tumor growth (FIG. 8) compared to vehicle and αPD-L1 treated mice.

A sandwich ELISA (anti-Fc capture antibody followed by anti-IL15 detection antibody) was used to measure IL15-Fc content in the blood (D3, D6 and D20; 1, 4 and 18 days post ACT, respectively) of mice injected with Deep IL-15 Primed PMEL T cells alone or in combination with αPD-L1. The results are shown in FIG. 8. Systemic IL15-Fc was quantified on D3 and D6, and was undetected at D20. Systemic exposure to IL15-Fc was unaffected by the addition of αPD-L1 to the treatment regimen.

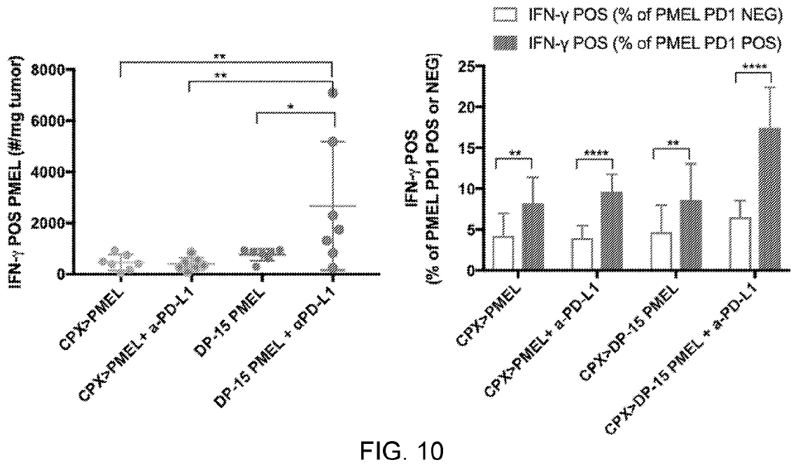

Combination of DEEP™ IL-15 Primed PMEL T cells with αPD-L1 resulted in more pronounced PMEL T cell activation in the tumor microenvironment, as measured by increased IFN-γ POS PMEL T cells within the tumor (FIG. 10). Additionally, higher percentages of IFN-γ POS PMEL T cells were detected among the PD1 positive PMEL T cells subset, consistent with an activation phenotype.

### Discussion

In this study, we evaluated the anti-tumor activity of a single-dose of 5×10⁶ DEEP™ IL-15 Primed PMEL T cells alone or in combination with PD-L1 blockade in B16-F10 tumor-bearing mice. The control groups included vehicle and αPD-L1 single-agent treated mice. Treatment with αPD-L1 alone did not result in delayed tumor growth compared to vehicle. Adoptive cell transfer (ACT) with DEEP™ IL-15 Primed PMEL T cells resulted in statistically significant tumor growth inhibition and increased survival compared to either vehicle or αPD-L1 control. Combination of DEEP™ IL-15 Primed PMEL T cells with αPD-L1 resulted in a statistically significant improvement of anti-tumor activity and increased survival compared to the individual agents. Importantly, DEEP™ IL-15 Primed PMEL T cell ACT with or without PD-L1 blockade was well tolerated, with only a minor and transient, toxicologically non relevant body weight loss relative to the original weights before treatment initiation. A reduced weight gain compared to vehicle or αPD-L1 treated mice was likely attributable to reduced tumor growth. Finally, addition of the αPD-L1 blocking antibody to DEEP™ IL-15 Primed PMEL T cells did not increase systemic IL15-Fc levels. Taken together, these findings suggest that the combination of DEEP™ IL-15 Primed T cell ACT and PD-L1 blockade augments anti-tumor efficacy without eliciting major toxicities in mice.

In conclusion, combining DEEP™ IL-15 Primed T cell ACT with PD-L1 blockade augments anti-tumor efficacy without eliciting major toxicities in mice. Treatment with DEEP™ IL-15 Primed PMEL T cells resulted in a statistically significant tumor growth inhibition and improved survival compared to either vehicle control or single agent αPD-L1, including 3/10 CRs but no TFS. Combination of DEEP™ IL-15 Primed PMEL T cell ACT with PD-L1 blockade induced a statistically significant improvement of tumor growth inhibition compared to vehicle control or single agents. Combination of DEEP™ IL-15 Primed PMEL T cell ACT with αPD-L1 improved median survival compared to both single agents and resulted in 10/10 CRs and 6/10 TFS at study termination (D76). All treatment groups were well tolerated. DEEP™ IL-15 Primed PMEL T cell ACT with or without PD-L1 blockade was accompanied by only a minor and transient, toxicologically non relevant body weight loss relative to the original weights before treatment initiation. DEEP™ IL-15 Primed PMEL T cell ACT alone or in combination with αPD-L1 resulted in a reduced weight gain compared to vehicle or αPD-L1 treated mice. This observation was likely attributable to reduced tumor growth. Addition of αPD-L1 to DEEP′ IL-15 Primed PMEL T cell therapy did not affect systemic exposure to IL15-Fc.

### Example 3: DEEP™ IL-12 Combined with Checkpoint Inhibition Boosts Anti-Tumor Durability

Surface-tethered IL-12 was evaluated for the ability to enhance anti-tumor response to immune checkpoint inhibition. An IL-12 tethered fusion (IL-12 TF; chM1Fab-IL-12) was constructed to enable tethering of IL-12 to PMEL T cells in accordance with PCT International Publication Nos. WO 2019/010219 and WO 2019/010222, each incorporated herein by reference in its entirety.

To evaluate the ability of a combination comprising a tumor-specific cell therapy tethered with an IL-12 TF and a checkpoint inhibitor to enhance anti-tumor efficacy the tumor growth inhibition and tumor microenvironment effects were evaluated using the PMEL/B16-F10 T cell therapy cancer model. Briefly, C57BL/6J mice were inoculated intradermally with 400,000 B16-F10 melanoma cells. Separately CD8 T cells were isolated from Pmel-1 mice, which express a T cell receptor specific for the mouse gp100 antigen in B16-F10 melanoma cells. The mouse CD8 T cells were activated for two days using antibodies against mouse CD3 and CD28 receptors, and then expanded in the presence of IL-21 for two days. Briefly, isolated CD8 T cells were incubated in full medium (containing RPMI 1640, 10% FBS, insulin-transferrin-selenium, penicillin/streptomycin, and 50 uM beta-mercaptoethanol) in Nunc HighBind plates that were previously coated with antibodies against mouse CD3 and CD28 receptors; the CD3 and CD28 antibodies were coated at concentrations of 0.5 ug/mL and 5 ug/mL, respectively. After approximately 24 hr incubation on the antibody-coated plates IL-2 and IL-7 were added to a final concentrations of 20 ng/mL and 0.5 ng/mL respectively. After a second day of incubation, cells were recovered from the antibody-coated plates and diluted to a density of approximately 200,000 cells/mL into medium containing IL-21 at a final concentration of 20 ng/mL. After one day expansion in IL-21 cells were diluted again to a density of approximately 200,00 cells/mL into medium containing IL-21 at a final concentration of 20 ng/mL. Cells were then recovered, washed, and incubated with IL-12 TF (chM1Fab-scIL-12p70) at a concentration of 125 nM for 30 min at 37 C, followed by three washes to remove unbound IL-12 TF. Cell were resuspended in HBSS and adoptively transferred (5×10⁶ cell/mouse) to the B16-F10 tumor-bearing mice by tail vein injection. As controls, mice were also treated with HBSS or Pmel T cells alone. One day prior to the adoptive cell transfer the tumor-bearing mice were treated with 4 mg cyclophosphamide. Anti-PD-L1 antibody (clone 10F.9G2) was administered intraperitoneally two times per week for six weeks at a dose of 200 ug per dose. Second and third doses of PMEL T cells tethered with the IL-12 TF were administered 14 and 28 days after the first cell dose. These second and third doses were administered in the absence of cyclophosphamide treatment (i.e. cyclophosphamide was only included prior to the first cell dose). PCT International Publication No. WO 2019/010219 demonstrated that multiple doses of tumor-specific T cells loaded with an IL-12 TF—but not multiple doses of the tumor-specific T cells alone—improved anti-tumor survival compared with a single T cell dose. Thus, this study evaluated multiple doses for only T cells loaded with an IL-12 TF.

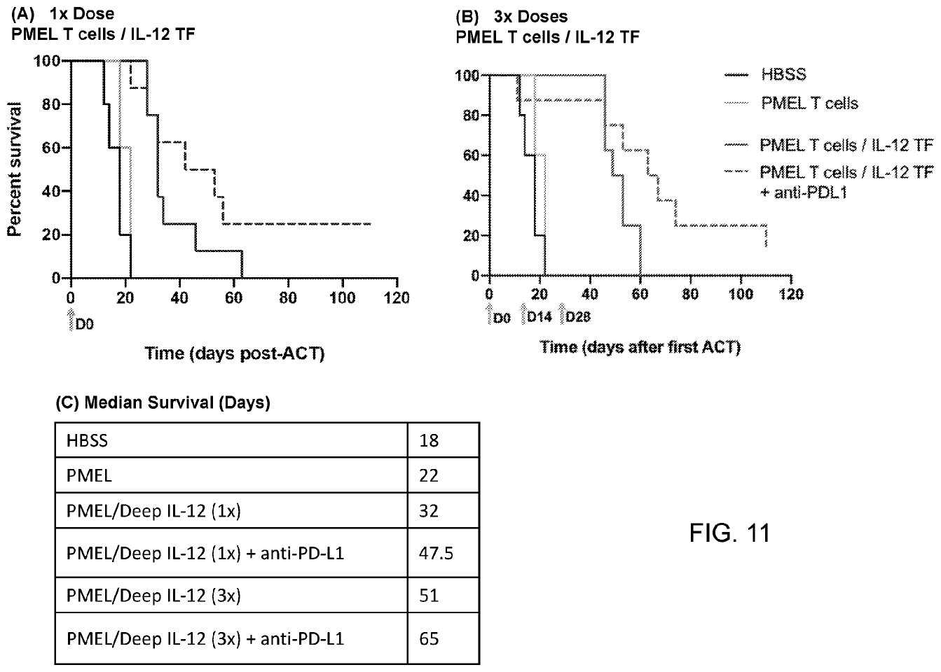

Survival analysis in FIG. 11 demonstrates that the IL-12 TF enhances antitumor activity compared to PMEL T cells alone, and multiple doses of the PMEL T cells tethered with the IL-12 TF further improved anti-tumor efficacy. In each case—in combination with single or multiple doses of PMEL T cells tethered with the IL-12 TF-combination with the anti-PD-L1 checkpoint inhibitor further improved anti-tumor efficacy. This is characterized by both an improvement in median survival and an increase in the number of durable long-term survivors.

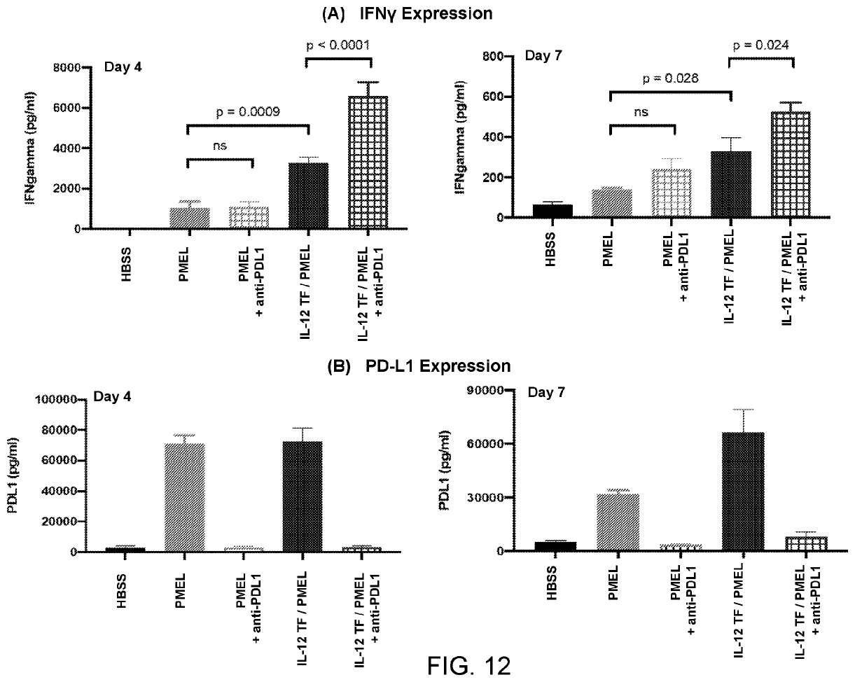

To evaluate effects of the combination immunotherapy in the tumor microenvironment, a separate study was conducted to evaluate biomarkers of inflammation in the tumor following the combination immunotherapy. IFNg is a key cytokine produced by IL-12 that mediates much of the pro-inflammatory effects of IL-12. Mice were treated with PMEL T cells and PMEL T cells tethered with an IL-12 TF (chM1Fab-scIL-12) as described above and then were sacrificed seven days after treatment and analyzed for IFNg and PD-L1 expression in the tumor by ELISA or Luminex. Statistical significance was assessed using one-way analysis of variance followed by a Tukey post-test to compare all samples to each other. The PMEL T cells tethered with the IL-12 TF enhanced IFNg production compared to PMEL T cells alone and IFNg production was further enhanced by combining the IL-12 TF with anti-PD-L1 (FIG. 12). Anti-PD-L1 treatment did not enhance IFNg production when combined with PMEL T cells alone (FIG. 12). Without wishing to be bound by theory, it is believed that the combination is delivering unexpectedly enhanced anti-tumor efficacy at least in part by enhancing the pro-inflammatory effects of the IL-12 TF in the tumor. These effects were accompanied by a decrease in the apparent concentration of PD-L1 in the tumor. Without wishing to be bound by theory, it is further believed that the anti-PD-L1 antibody may bind to the same epitopes on the PD-L1 protein as the antibodies in the ELISA assay. We interpret this to suggest that the anti-PD-L1 antibody is decreasing the bioavailable concentration of PD-L1 in the tumor.

### Modifications

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A composition comprising:
a nucleated cell loaded with a plurality of protein clusters and/or immunostimulatory fusion molecules (IFMs); and
an inhibitor of a checkpoint inhibitor;
wherein each protein cluster comprises a plurality of therapeutic protein monomers reversibly cross-linked to one another via a plurality of biodegradable cross-linkers, wherein the protein cluster has a size between 30 nm and 1000 nm in diameter measured by dynamic light scattering, wherein the cross-linker degrades, after administration into a subject in need thereof, under physiological conditions so as to release the therapeutic protein monomers from the protein cluster, wherein optionally the protein cluster further comprises a surface modification such as polycation so as to allow the protein cluster to associate with the nucleated cell;
wherein each IFM is engineered to contain an immunostimulatory cytokine molecule and a targeting moiety (e.g., an antibody or an antigen-binding fragment thereof) having an affinity to an antigen on the surface of the nucleated cell, wherein the immunostimulatory cytokine molecule is operably linked to targeting moiety.

2. The composition of claim 1, wherein the nucleated cell is from a population of T cells that have been enriched or trained to possess specificity against one or more tumor-associated antigens (TAAs).

3. The composition of claim 1, wherein the nucleated cell is substantially purified.

4. The composition of claim 1, wherein the nucleated cell is autologous to a subject in need of the composition.

5. The composition of claim 1, wherein the therapeutic protein monomers include one or more cytokine molecules and optionally one or more costimulatory molecules, wherein:
(i) the one or more cytokine molecules are selected from IL-15, IL-2, IL-7, IL-10, IL-12, IL-18, IL-21, IL-23, IL-4, IL-1alpha, IL-1beta, IL-5, IFNgamma, TNFa, IFNalpha, IFNbeta, GM-CSF, or GCSF; and
(ii) the one or more costimulatory molecules are selected from CD137, OX40, CD28, GITR, VISTA, anti-CD40 antibody, or CD3.

6. The composition of claim 1, wherein in the IFM, the immunostimulatory cytokine molecule is selected from one or more of IL-15, IL-2, IL-6, IL-7, IL-12, IL-18, IL-21, IL-23, or IL-27 or variant forms thereof, and wherein the antigen is selected from one or more of CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR.

7. The composition of claim 1, wherein the checkpoint inhibitor is one or more of PD-1, PD-L1, LAG-3, TIM-3, or CTLA-4.

8. The composition of claim 1, wherein the inhibitor of the checkpoint inhibitor is an antibody or antigen-binding fragment thereof that binds and neutralizes or inhibits the checkpoint inhibitor.

9. A method for providing cancer immunotherapy, comprising:
administering to a patient in need thereof a plurality of nucleated cells loaded with a plurality of protein clusters and/or immunostimulatory fusion molecules (IFMs); and
administering to the patient an inhibitor of a checkpoint inhibitor;
wherein each protein cluster comprises a plurality of therapeutic protein monomers reversibly cross-linked to one another via a plurality of biodegradable cross-linkers, wherein the protein cluster has a size between 30 nm and 1000 nm in diameter measured by dynamic light scattering, wherein the cross-linker degrades, after administration into a subject in need thereof, under physiological conditions so as to release the therapeutic protein monomers from the protein cluster, wherein optionally the protein cluster further comprises a surface modification such as polycation so as to allow the protein cluster to associate with the nucleated cell;
wherein each IFM is engineered to contain an immunostimulatory cytokine molecule and a targeting moiety (e.g., an antibody or an antigen-binding fragment thereof) having an affinity to an antigen on the surface of the nucleated cell, wherein the immunostimulatory cytokine molecule is operably linked to targeting moiety.

10. The method of claim 9, wherein the nucleated cell is substantially purified.

11. The method of claim 9, wherein the nucleated cell is autologous to a subject in need of the composition.

12. The method of claim 9, further comprising administering the nucleated cell and the inhibitor of checkpoint inhibitor separately.

13. The method of claim 9, further comprising administering the nucleated cell and the inhibitor of checkpoint inhibitor sequentially.

14. The method of claim 9, wherein the nucleated cell is from a population of T cells that have been enriched or trained to possess specificity against one or more tumor-associated antigens (TAAs).

15. The method of claim 9, wherein the therapeutic protein monomers include one or more cytokine molecules and optionally one or more costimulatory molecules, wherein:
(i) the one or more cytokine molecules are selected from IL-15, IL-2, IL-7, IL-10, IL-12, IL-18, IL-21, IL-23, IL-4, IL-1alpha, IL-1beta, IL-5, IFNgamma, TNFa, IFNalpha, IFNbeta, GM-CSF, or GCSF; and
(ii) the one or more costimulatory molecules are selected from CD137, OX40, CD28, GITR, VISTA, anti-CD40 antibody, or CD3.

16. The method of claim 9, wherein in the IFM, the immunostimulatory cytokine molecule is selected from one or more of IL-15, IL-2, IL-6, IL-7, IL-12, IL-18, IL-21, IL-23, or IL-27 or variant forms thereof, and wherein the antigen is selected from one or more of CD45, CD4, CD8, CD3, CD11a, CD11b, CD11c, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD16, CD56, CD137, OX40, or GITR.

17. The method of claim 9, wherein the checkpoint inhibitor is one or more of PD-1, PD-L1, LAG-3, TIM-3, or CTLA-4.

18. The method of claim 9, wherein the inhibitor of the checkpoint inhibitor is an antibody or antigen-binding fragment thereof that binds and neutralizes or inhibits the checkpoint inhibitor.
